SEQUENCE LISTING (1 sequence)

SEQ ID NO: 1 - DNA, 714 bp, Artificial
  PCV2b&d-ConCP
gccgccaccatgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaagaaaaccacagtcagaacgccctcctgggcggtggacatgatgagatttaatattaatgattttcttcccccaggagggggctcaaaccccctctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtccttgataggacaatcgattacttccaacccaataacaaaagaaatcagctctggctgagactacaaactactggaaatgtagaccacgtaggcctcggcactgcgttcgaaaacagtatatacgaccaggactacaatatccgtgtaaccatgtatgtacaattcagagaatttaatcttaaagaccccccacttaaccctaaatga

(19) United States
(12) Patent Application Publication
(10) Pub. No.: US 2025/0018022 A1
(43) Pub. Date: Jan. 16, 2025

(54) NOVEL RECOMBINANT PORCINE CIRCOVIRUS TYPE 2 PROTEIN AND USE THEREOF

(71) Applicant: INNOVAC INC., Chuncheon-si (KR)

(72) Inventors: Tae Wook HAHN, Seoul (KR); Ki Ju KIM, Chuncheon-si (KR); Kyu Sung CHOI, Chuncheon-si (KR)

(73) Assignee: INNOVAC INC., Chuncheon-si (KR)

(21) Appl. No.: 18/711,502

(22) PCT Filed: Jun. 14, 2022
(86) PCT No.: PCT/KR2022/008361

§ 371 Date: May 17, 2024

(30) Foreign Application Priority Data
Nov. 19, 2021 (KR) 10-2021-0159907

(51) Int. Cl. A61K 39/12; A61P 37/04; C07K 14/005

(52) U.S. Cl. CPC A61K 39/12; A61P 37/04; C07K 14/005; C12N 2750/10022; C12N 2750/10033; C12N 2750/10071

(57) ABSTRACT

The present invention relates to a novel recombinant porcine circovirus type 2 protein and use thereof and, more specifically, to: a novel recombinant porcine circovirus type 2 protein; and a vaccine composition, an immunogenic composition, and a feed composition, comprising same. It was confirmed that the novel recombinant porcine circovirus type 2 protein according to the present invention showed excellent antibody titers against both PCV2b and type 2d. This means that when the recombinant porcine circovirus type 2 protein of the present invention is used as a vaccine, porcine circovirus-related diseases can be effectively prevented by cross-protecting both PCV2b and PCV2d genotypes, and thus the porcine circovirus type 2 protein of the present invention can be used in various ways in the field of pig farming.

### TECHNICAL FIELD

The present invention relates to a novel recombinant porcine circovirus type 2 protein and use thereof and more particularly, to a novel recombinant porcine circovirus type 2 protein; and a vaccine composition, an immunogenic composition, and a feed composition, including the same.

### BACKGROUND ART

Porcine circovirus type 2 (PCV2) is a circular single-stranded DNA virus belonging to the Circoviridae family and Circovirus genus, and a virus that has no hemagglutination ability, is inactivated under acidic conditions (pH 3) or chloroform, and is stable even at a high temperature (70° C., 15 min). PCV2 is a main cause of postweaning multisystemic wasting syndrome (PMWS), which is causing a lot of damage to the pig farming industry worldwide, and causes various lesions, such as porcine dermatitis and nephropathy syndrome (PDNS), porcine respiratory disease complex (PRDC), reproductive disorders, and enteritis. Recently, these diseases have been collectively named porcine circovirus associated diseases (PCVAD).

PCV2 is mainly known to be infected by direct contact through the oral or nasal cavity, or through feces and urine. In addition, it was confirmed that PCV2 antigens were detected even in semen and organs of miscarriage and stillborn fetuses to be vertically propagated. After PCV2 is infected to pigs, the primary proliferation of the virus is estimated to occur in macrophages and dendritic cells in lymphoid tissues such as tonsils or lymph nodes, and the proliferated virus spreads to systemic lymphatic organs through lymph and blood to cause lymphoid depletion and peripheral blood lymphopenia. Therefore, it is understood that PCV2 infected in pigs causes immunosuppression due to lymphoid depletion of systemic lymphatic organs to facilitate infection by other viruses or secondary bacteria, thereby causing various diseases such as PMWS and PCVAD. In Korea, it was reported that the PCV2 antigen was detected in the intestinal tissue of 83.3% of 24 animals diagnosed with mixed infection of PCV2 and salmonellosis. In addition, the PCV2 antigens were detected within the lesions with granulomatous enteritis in the small intestine and large intestine of six piglets aged 40 to 60 days that showed diarrhea symptoms. In addition, only when there is no systemic lymphoid tissue lesion in pigs and lymphoid depletion is shown in gut-associated lymphatic tissue (GALT) along with enteritis lesions, there was proposed to be named Enteritis associated with porcine circovirus type 2 (PCV2) (EAPC). However, compared to other diseases included in PCVAD, research on EAPC is still relatively lacking.

### DISCLOSURE

### Technical Problem

Accordingly, the present inventors completed the present invention by developing recombinant proteins for porcine circovirus types 2b and 2d and confirming their efficacy as a vaccine.

Therefore, an object of the present invention is to provide a nucleic acid encoding a recombinant porcine circovirus type 2 (PCV2) protein represented by a nucleotide sequence represented by SEQ ID NO: 1.

Another object of the present invention is to provide a recombinant porcine circovirus type 2 protein encoded by a nucleotide sequence represented by SEQ ID NO: 1.

Yet another object of the present invention is to provide a composition including a recombinant porcine circovirus type 2 protein encoded by a nucleotide sequence represented by SEQ ID NO: 1.

Still another object of the present invention is to provide a method for preventing or treating infectious diseases of porcine circovirus type 2 including administering a porcine with a recombinant porcine circovirus type 2 protein encoded by a nucleotide sequence represented by SEQ ID NO: 1.

### Technical Solution

An aspect of the present invention provides a nucleic acid encoding a recombinant porcine circovirus type 2 protein represented by a nucleotide sequence represented by SEQ ID NO: 1.

In addition, another aspect of the present invention provides a recombinant porcine circovirus type 2 protein encoded by a nucleotide sequence represented by SEQ ID NO: 1.

In addition, yet another aspect of the present invention provides a vaccine composition for preventing or treating infectious diseases of porcine circovirus type 2 including a recombinant porcine circovirus type 2 protein encoded by a nucleotide sequence represented by SEQ ID NO: 1.

In addition, yet another aspect of the present invention provides an immunogenic composition for porcine circovirus type 2 including a recombinant porcine circovirus type 2 protein encoded by a nucleotide sequence represented by SEQ ID NO: 1.

In addition, yet another aspect of the present invention provides a feed composition for preventing or improving infectious diseases of porcine circovirus type 2 including a recombinant porcine circovirus type 2 protein encoded by a nucleotide sequence represented by SEQ ID NO: 1.

In addition, yet another aspect of the present invention provides a feed additive composition for preventing or improving infectious diseases of porcine circovirus type 2 including a recombinant porcine circovirus type 2 protein encoded by a nucleotide sequence represented by SEQ ID NO: 1.

In addition, still another aspect of the present invention provides a method for preventing or treating infectious diseases of porcine circovirus type 2 including administering a porcine with a recombinant porcine circovirus type 2 protein encoded by a nucleotide sequence represented by SEQ ID NO: 1.

### Advantageous Effects

According to the present invention, it was confirmed that the novel recombinant porcine circovirus type 2 protein showed excellent antibody titers against both PCV2b and 2d types. This means that when the recombinant porcine circovirus type 2 protein of the present invention is used as a vaccine, porcine circovirus-related diseases can be effectively prevented by completely cross-protecting both PCV2b and PCV2d genotypes, and thus the porcine circovirus type 2 protein of the present invention can be used in various ways in the field of pig farming.

### DESCRIPTION OF DRAWINGS

FIG. 1 is a diagram showing a result of Pierce BCA Protein Assay for quantifying vaccine antigen concentrations.

FIG. 2 is a diagram showing a result of measuring PCV2b-specific IgG antibody levels in guinea pigs.

FIG. 3 is a diagram showing a result of measuring PCV2d-specific IgG antibody levels in guinea pigs.

FIG. 4 is a diagram showing a result of examining interferon gamma release amounts in guinea pig PBMC.

FIG. 5 is a diagram showing a result of confirming neutralizing antibody titers against PCV2b in guinea pigs.

FIG. 6 is a diagram showing a result of confirming neutralizing antibody titers against PCV2d in guinea pigs.

FIG. 7 is a diagram showing a result of measuring PCV2b-specific IgG antibody levels in pigs.

FIG. 8 is a diagram showing a result of measuring PCV2d-specific IgG antibody levels in pigs.

FIG. 9 is a diagram showing a result of measuring PCV2b-specific IgA antibody levels in pigs.

FIG. 10 is a diagram showing a result of measuring PCV2d-specific IgA antibody levels in pigs.

FIG. 11 is a diagram showing a result of examining interferon gamma release amounts in pig PBMC.

FIG. 12 is a diagram showing a result of confirming neutralizing antibody titers against PCV2b in pigs.

FIG. 13 is a diagram showing a result of confirming neutralizing antibody titers against PCV2d in pigs.

FIG. 14 is a diagram showing a result of measuring PCV2d viremia in pig blood.

FIG. 15 is a diagram showing a result of measuring PCV2d DNA loads in pig lungs and lymph nodes.

FIG. 16 is a diagram showing a result of measuring PCV2d DNA loads in pig Nasal and Rectal swab.

FIG. 17 is a diagram showing a result of visual observation of gross lesions in pig lungs and lymph nodes after challenge.

FIG. 18 is a diagram showing a result of quantifying lymphoid hyperplasia around the bronchus of pig lungs.

FIG. 19 is a diagram showing a result of confirming the number of lymphoid nodules around the bronchus in a pig lung tissue section.

FIG. 20 is a diagram illustrating a result of immunohistochemical staining to identify lymphoid nodules around the bronchus in a pig lung tissue section.

FIG. 21 is a diagram showing a result of confirming inflammatory cell infiltration observed around blood vessels and bronchus in the pig lung parenchyma.

FIG. 22 is a diagram showing a result of immunohistochemical staining for PCV2 in pig lung (Ln, Lymphatic nodule: a, alveolus; b, bronchiole).

FIG. 23 is a diagram showing a result of detecting PCV2 antigens in a pig lung sample through immunohistochemical staining.

FIG. 24 is a diagram showing a result of measuring the number of secondary lymphatic nodules in pig lymph nodes.

FIGS. 25 to 27 are diagrams showing results of detecting PCV2 antigens in pig lymph nodes through immunohistochemical staining.

FIG. 28 is a diagram showing a result of measuring body weights of piglets by week of age.

### BEST MODE OF THE INVENTION

Hereinafter, the present invention will be described in detail.

According to an aspect of the present invention, the present invention provides a nucleic acid encoding a recombinant porcine circovirus type 2 protein represented by a nucleotide sequence represented by SEQ ID NO: 1; and a recombinant porcine circovirus type 2 protein encoded by the nucleic acid.

In the present invention, porcine circovirus type 2 (PCV2) is a circular single-stranded DNA virus belonging to the Circovirus genus, and a virus that has no hemagglutination ability, and is inactivated under acidic conditions (pH 3) or chloroform, and stable even at a high temperature (70° C., 15 min).

In an embodiment of the present invention, the nucleic acid is preferably recombined by genes of porcine circovirus types 2b and 2d, and more preferably. recombined by genes of PCV types 2b and 2d as a single sequence using the Baculovirus Express system, but is not limited thereto.

In an embodiment of the present invention, the nucleic acid preferably includes a sequence encoding a decoy epitope.

In the present invention, the decoy epitope is an immunodominant region consisting of 169-180 residues (Cap (169-180)), and refers to a site that is exposed to the surface of a Cap protein monomer, but is buried in a virus particle or virus-like particle to avoid humoral immunity.

The scope of the present invention includes variants of the nucleotide sequence. Specifically, the gene has a sequence homology of 70% or more, more preferably 80% or more, even more preferably 90% or more, and most preferably 95% or more with a nucleotide sequence represented by SEQ ID NO. 1, and means a sequence that exhibits substantially the same physiological activity as the nucleotide sequence represented by SEQ ID NO. 1. The “% of sequence homology” with a polynucleotide is confirmed by comparing two optimally aligned sequences with a comparison region, and a part of a polynucleotide sequence in the comparison region may include addition or deletion (i.e., gap) compared to a reference sequence (without including addition or deletion) for an optimal alignment of the two sequences.

In addition, the recombinant porcine circovirus type 2 protein includes a protein encoded by the nucleotide sequence represented by SEQ ID NO: 1 and functional equivalents of the protein.

The “functional equivalent” refers to a protein which has a sequence homology (that is, identity) of at least 80% or more, preferably 90%, more preferably 95% or more with the protein encoded by SEQ ID NO: 1, for example, a sequence homology of 80%, 81%, 82%, 83%, 84%, 85%, 86%, 87%, 88%, 89%, 90%, 91%, 92%, 93%. 94%, 95%, 96%, 97%, 98%, 99%, and 100% as a result of addition, substitution or deletion of amino acids, and exhibits substantially homogeneous physiological activity with the protein encoded by SEQ ID NO: 1. In the present specification, the sequence homology and homogeneity are defined as a percentage of amino acid residues of a candidate sequence to the amino acid sequence encoded by SEQ ID NO: 1 after aligning the amino acid sequence encoded by SEQ ID NO: 1 and the candidate sequence and introducing gaps. If necessary, conservative substitution is not considered as a part of sequence homogeneity in order to obtain the maximum percentage sequence homogeneity. An N-terminal, a C-terminal or internal extension, deletion or insertion of the amino acid sequence encoded by SEQ ID NO: 1 is not construed as a sequence affecting sequence homogeneity or homology.

In addition, the sequence homogeneity may be determined by general standard methods used for comparing similar portions of amino acid sequences of two proteins. A computer program such as BLAST or FASTA aligns the two proteins so as to be optimally matched with each amino acid (according to a full-length sequence of one or two sequences, or a predicted portion of one or two sequences). The program provides a default opening penalty and a default gap penalty and provides a scoring matrix such as PAM250 (standard scoring matrix) which may be used in association with the computer program. For example, the percentage homogeneity may be calculated as follows. The total number of identical matches is multiplied by 100 and then divided into a sum of the length of a longer sequence in a corresponding matched span and the number of gaps introduced into the longer sequence to align the two sequences.

Here, the “substantially homogeneous physiological activity” refers to the activity that induces immunity against PCV types 2b and 2d in a subject when applied to the subject. The range of “functional equivalents” of the present invention includes derivatives in which some chemical structures of a peptide are modified while maintaining a basic skeleton of the peptide encoded by SEQ ID NO: 1 and the activity of inducing immunity against PCV types 2b and 2d. For example, the range of the functional equivalents includes structural modifications for changing the stability, storage, volatility, solubility or the like of the peptide.

According to another aspect of the present invention, the present invention provides a composition for preventing or treating infectious diseases of porcine circovirus type 2 including a recombinant porcine circovirus type 2 protein encoded by a nucleotide sequence represented by SEQ ID NO: 1. The composition may be a vaccine composition or an immunogenic composition.

In an embodiment of the present invention, the porcine circovirus type 2 is preferably porcine circovirus type 2b or 2d. Specifically, it is experimentally confirmed that the porcine circovirus type 2 protein of the present invention has significantly high antibody titers against both PCV types 2b and 2d, which means that the protein of the present invention cross-protects two genotypes PCV2b and PCV2d.

In an embodiment of the present invention, the infectious disease of porcine circovirus type 2 is preferably at least one disease selected from the group consisting of porcine reproductive and respiratory syndrome (PRRS), Glasser's disease, streptococcal meningitis, salmonellosis, postweaning colibacillosis, dietetic hepatosis, and suppurative bronchopneumonia, but is not limited thereto.

In an embodiment of the present invention, the composition may be administered preferably intravenously, intramuscularly, intranasally, intra-articularly, intra-synovially, intrathecally, intrahepatically, intralesionally or intracranially, and more preferably, intramuscularly or intranasally.

In the present invention, the vaccine is a veterinary vaccine containing an antigenic substance and is administered for inducing specific, active or passive immunity against PCV2. In an example of the present invention, it is confirmed that the recombinant porcine circovirus type 2 protein of the present invention exhibits excellent antibody titers against PCV types 2b and 2d, and thus may be useful in preventing diseases caused by PCV2.

The vaccine composition according to the present invention may be administered in an immunologically effective amount. The “immunologically effective amount” refers to an amount enough to show a preventive effect against PCV2-related diseases and an amount that does not cause side effects or serious or excessive immune responses. The exact administered concentration depends on a specific immunogen to be administered, and may be easily determined by those skilled in the art according to factors well known in the medical field, such as the age, body weight, health, sex, sensitivity of a subject to a drug, administration route, and administration method of a vaccination subject, and administered once or several times.

The vaccine composition according to the present invention may include one or more immune boosters, excipients, or carriers suitable for constituting the vaccine composition, in addition to the recombinant porcine circovirus type 2 protein as an active ingredient.

The adjuvant that may be included in the vaccine composition of the present invention refers to a substance that enhances the immune responses of an injected animal, and a plurality of different immune boosters is known to those skilled in the art. The immune boosters include Freund's complete and incomplete immune boosters, vitamin E, nonionic blocking polymers, muramyl dipeptide, Quil A, mineral oil and non-mineral oil, Carbopol, water-in-oil emulsion immune boosters, etc., but are not limited thereto.

The carriers that may be included in the vaccine composition of the present invention are known to those skilled in the art and include proteins, sugars, etc., but are not limited thereto. The carrier may be an aqueous or non-aqueous solution, a suspension and an emulsion. Examples of the non-aqueous carrier may include propylene glycol, polyethylene glycol, edible oils such as olive oil, and injectable organic esters such as ethyl oleate. The aqueous carrier includes water, alcohol/aqueous solutions, emulsions or suspensions, including saline solutions and buffered media. Parenteral carriers include a sodium chloride solution, Ringer's dextrose, dextrose and sodium chloride, lactated Ringer's solution or fixed oils. Carriers for intravenous injection include electrolyte supplements, liquid and nutritional supplements, such as carriers based on Ringer's dextrose.

The vaccine composition of the present invention may further include preservatives and other additives, for example, antimicrobial agents, antioxidants, chelating agents, inert gases, and the like. The preservatives include formalin, thimerosal, neomycin, polymyxin B, amphotericin B, and the like. The vaccine composition of the present invention may include one or more suitable emulsifiers. such as Span or Tween. In addition, the vaccine composition of the present invention may include a protecting agent, and protecting agents known in the art may be used without limitation, and may include Lactose (LPGG) or Trehalose (TPGG), but are not limited thereto.

According to yet another aspect of the present invention, the present invention provides a feed composition for preventing or improving infectious diseases of porcine circovirus type 2 including a recombinant porcine circovirus type 2 protein encoded by a nucleotide sequence represented by SEQ ID NO: 1. The composition may be a feed composition or a feed additive composition.

In the present invention, the feed refers to any natural or artificial constituted meal eaten by animals, one meal, or ingredients of one meal, and the feed containing the recombinant porcine circovirus type 2 protein according to the present invention as an active ingredient may be prepared in various types of feed known in the art, and preferably may include concentrated feed, roughage and/or special feed, but is not limited thereto.

In the present invention, the feed additives mean substances added to the feed for the purpose of various effects, such as alleviation of disease symptoms in animals, nutrient supplementation and weight loss prevention, improvement of digestibility of fiber in feed, oil quality improvement, reproduction disorder prevention and conception rate improvement, and summer high temperature stress prevention.

The feed composition and the feed additive composition of the present invention correspond to supplementary feed under the Feed Management Act, and may further include mineral preparations such as sodium bicarbonate, bentonite, magnesium oxide, and complex minerals, mineral preparations, which are trace minerals such as zinc, copper, cobalt, and selenium, vitamins such as carotene, vitamins A, D, E, nicotinic acid, vitamin B complex, protected amino acid supplements such as methionine and lysine, protected fatty acid preparations such as fatty acid calcium salts, active bacterial and yeast agents such as probiotics (lactic acid bacteria), yeast cultures, and mold fermented products, and the like.

Among the feeds, the concentrated feed is a by-product obtained by refining seed fruits including grains such as wheat, oats and corn and grains, and includes bran including rice bran, bran, barley bran, etc., residues such as seed cakes, which are by-products obtained from oil extraction of beans, oil, sesame, linseed, and coco palms, and residual starches as a main ingredient of starch residues, which are the remainder after removing starch from sweet potatoes and potatoes, fish soluble, which is a concentrate of fish meal, fish residues, and fresh liquids obtained from fish, animal feeds such as meat meal, blood meal, feather meal, skim milk powder, dried whey, which is obtained by drying whey as the balance when preparing cheese from milk and casein from skim milk, yeast, chlorella, and seaweed, but is not limited thereto.

Among the feeds, the roughage includes raw grass feed such as wild grass, grass, and green cutting, root vegetables such as turnips for feed, beets for feed, and Lutherbearer, a type of turnip, silage, which is stored feed made by filling a silo with green grass, green crops, and grains, and fermenting the silo with lactic acid, dried hay made by cutting and drying wild grass and grass, straw of breeding crops, and leaves of leguminous plants, but is not limited thereto. The special feed includes mineral feeds such as oyster shells and rock salt, urea feeds such as urea or its derivative, diureidisobutane, feed additives which are substances supplemented with ingredients that tend to be insufficient when only natural feed raw materials are mixed, or added in a small amount to the formulated feed to improve the storage of feeds, and dietary supplements, but is not limited thereto.

According to still another aspect of the present invention, the present invention provides a method for preventing or treating infectious diseases of porcine circovirus type 2 including administering a porcine with a recombinant porcine circovirus type 2 protein encoded by a nucleotide sequence represented by SEQ ID NO: 1.

In an embodiment of the present invention, the recombinant porcine circovirus type 2 protein may be administered preferably intravenously, intramuscularly, intranasally, intra-articularly. intra-synovially, intrathecally, intrahepatically, intralesionally or intracranially, and more preferably, intramuscularly or intranasally, but is not limited thereto.

The method for preventing or treating infectious diseases of porcine circovirus type 2 of the present invention preferably increases in-vivo immune responses by administering the recombinant porcine circovirus type 2 protein, and more specifically, preferably controls antibody titers and the expression of immune response-related factors.

Duplicated contents are omitted in consideration of the complexity of the present specification, and terms not defined otherwise in the present specification have the meanings commonly used in the art to which the present invention pertains.

### Modes of the Invention

Hereinafter, the present invention will be described in more detail with reference to Examples. These Examples are just illustrative of the present invention, and it will be apparent to those skilled in the art that it is not interpreted that the scope of the present invention is limited to these Examples.

### EXPERIMENTAL EXAMPLES

### Experimental Example 1. Preparation of PCV2b&d-ConCP Vaccine

Among antigens of Innocirco vaccine (hereinafter Innocirco) developed by Innovac Co., Ltd., a recombinant Porcine circovirus 2b, 2d Consensus Capsid Protein (hereinafter PCV2b&d-ConCP) was prepared using the Baculovirus Express system. The prepared PCV2b&d-ConCP was recombined by genes of PCV types 2b and 2d into one sequence, and the PCV2b&d-ConCP was represented by a nucleotide sequence represented by SEQ ID NO: 1.

### Experimental Example 2. PCV2b&d-ConCP Vaccine Efficacy Evaluation Test in Guinea Pigs

### 2-1. Design and Schedule of Test

300 to 350 g guinea pigs were used as a test subject, and test groups and vaccine antigen concentrations used in the test were designed as shown in Table 1. The vaccination dose (½ dose of pig) and the vaccination route (thigh muscle vaccination) of guinea pig were set with reference to the standards of National Lot Release test. The test schedule was performed as shown in Table 2.

TABLE I

| | | Vaccine | | | |
|---|---|---|---|---|---|
| Group | | antigen | Vaccination | Subject | Vaccination |
| No. | Group name | concentration | dose | number | route |
| G4 | PCV2b& | 5 μg/mL/dose | 1 mL | 5 | Intramuscular |
| | d-ConCP | | | | vaccination |
| G5 | Bac-wt | 5 μg/mL/dose | 1 mL | 5 | Intramuscular |
| | | | | | vaccination |
| G6 | Control | — | 1 mL | 3 | Intramuscular |
| | (PBS) | | | | vaccination |

TABLE 2

| | Days | |
|---|---|---|
| | postvaccination | |
| No. | (DPV) | Performance detail |
| 1 | 0 | (Jugular vein) blood collection and vaccination |
| 3 | 14 | (Jugular vein) blood collection |
| 3 | 28 | (Jugular vein) blood collection |
| 4 | 42 | Euthanasia after (heart) blood collection |

### 2-2. Quantification of Vaccine Antigen Concentration and Preparation and Vaccination of Vaccine

### 1) Pierce BCA (Bicinchoninic Acid) Protein Assay

The vaccine antigen concentration was quantified according to the manufacturer's manual of the Pierce BCA Protein Assay kit (ThermoFisher, Cat No. 23227, Lot No. VA294776D). 25 μL each of standard (working range=5 to 250 μg/mL) and sample were added to each microplate well, respectively. The BSA concentration for each standard was shown in Table 3.

TABLE 3

| | Volume of | | Final BSA |
|---|---|---|---|
| | Diluent | Volume and Source | Concentration |
| Vial | (μL) | of BSA (μL) | (μg/mL) |
| A | 700 | 100 of Stock | 250 |
| B | 400 | 400 of vial A dilution | 125 |
| C | 450 | 300 of vial B dilution | 50 |
| D | 400 | 400 of vial C dilution | 25 |
| B | 400 | 100 of vial D dilution | 5 |
| F | 400 | 0 | 0 = Blank |

200 μL of a working reagent (WR) was added to each well and thoroughly mixed in a plate shaker for 30 seconds. The mixture was covered with a plate cover and reacted at 37° C. for 30 minutes. The absorbance (OD) value was measured at a wavelength of 562 nm using an Epoch™ Microplate Spectrophotometer (BioTek, USA),

### 2) Vaccine Preparation and Vaccination

The concentration of the purified antigen was quantified through BCA Protein assay and then diluted in Phosphate Buffered Saline (PBS) so that the antigen concentration was 5 μg/mL/dose (½ dose of pig). MONTANIDE™ IMS1313 (Seppic, IMS1313 VG N) was used as an excipient and mixed with the antigen diluted in PBS at a ratio of 50:50 (v/v). 1 mL of the vaccine with a final antigen amount of 5 μg (½ dose of pig vaccination) was administered intramuscularly by each 0.5 mL separately into both sides of the guinea pig's thigh.

### 2-3. Guinea Pig Test Method

6-week-old female guinea pigs weighing 300 to 350 g (DOOYEOLBIOTECH, Korea) were used. The guinea pigs after an acclimatization period were set to week 0, and each test group was vaccinated with a total of 1 mL of the vaccine by 0.5 mL separately into each muscle of both thighs. A Control group (G6) was injected with PBS under the same conditions. At 14 days (14 DPV), 28 days (28 DPV), and 42 days (42 DPV) after vaccination, blood was collected from the jugular vein of all groups, and serum was separated and stored at −70° C.

### 2-4. Method for Analyzing PCV2b and PCV2d Antibody Levels in Guinea Pigs

### 1) Indirect ELISA

The purified PCV2b and PCV2d antigens were diluted in 0.05 M sodium bicarbonate buffer (pH 9.4) and dispensed at each 100 μL/well (100 ng) on a MaxiSorp 96-well plate and coated at 420 C. for 16 to 24 hours. The coating solution was removed and washed three times with 0.05% Phosphate Buffered Saline Tween (hereinafter, PBS-T, 0.05% Tween-20 in PBS) at each 300 μL/well. 1% Bovine Serum Albumin (BSA) was diluted in PBS (BSA in PBS), dispensed at each 200 μL/well into each well, and blocked for 2 hours at 37° C. The blocking solution was removed and washed three times with 0.05% PBS-T at each 300 μL/well. As a primary antibody. guinea pig serum samples were diluted at 1:1,000 in 0.1% BSA and reacted at 37° C. for 2 hours. The primary antibody was removed and washed three times with 0.05% PBS-T at each 300 μL/well. As a secondary antibody, a HRP-conjugated goat anti-Guinea pig IgG polyclonal antibody (Abcam, Cat No. ab7139) was diluted at 1:10,000 and added at each 100 μL/well, and then reacted at 37° C. for 1 hour. The secondary antibody was removed and washed three times with 0.05% PBS-T at each 300 μL/well. As a substrate, TetraMethyl-Benzidine (TMB, Surmodics, USA, Cat No. TMBW-1000-01, Lot No.TMBWW18) was added at each 100 μL/well, reacted in the dark for 5 minutes at room temperature, and then added with 2 N H₂SO₄ at each 100 μL/well, and then the reaction was stopped. The absorbance (OD) value was measured at a wavelength of 450 nm using an Epoch™ Microplate Spectrophotometer (BioTek, USA).

### 2) Examination of Neutralizing Antibody Titers

Test serum was non-assimilated at 56° C. for 30 minutes. Using a 96-well plate, the serum was diluted two-fold, dispensed with an equal amount of PCV2b or PCV2d (400 TCID₅₀/mL), and reacted at 37° C. for 1 hour. PCV-1 free porcine kidney (PK-15) cells were dispensed at 2×10⁴ cells/100 μL/well and incubated in an incubator for 3 days, and then Immunofluorescence assay (IFA) was performed as follows. The culture medium was removed, washed once with PBS, and the plate was dried at 37° C. for about 10 minutes. 100 μL/well of 80% acetone was dispensed to the dried plate and then fixed at 4° C. for 1 hour. The fixing solution was removed and washed once with PBS, and then Pig anti-PCV2 antiserum was diluted at 1:500, dispensed at each 100 μL/well, and reacted at 37° C. for 1 hour. After washing three times with PBS, a Goat anti-Pig IgG (h+I) FITC Conjugate (Bethyl, Cat No. A100-10SF, Lot No. A100-105F-20) was diluted at 1:200, dispensed at 100 μL/well, and reacted at 37° C. for 30 minutes. After washing three times with PBS, the plate was observed under a fluorescence microscope, and the reciprocal of the highest serum dilution that inhibited PCV2 proliferation was determined as neutralizing antibody titers.

### 2-5. Examination of Interferon Gamma (IFN-γ) Release Amount in Guinea Pig PBMCs

### 1) Preparation of Samples for Measuring Cytokine Release Amount (PBMC Separation)

4 mL of blood and 4 mL of PBS were mixed in a conical tube, and then Ficoll (GE Healthcare, Cat No. 17-1440-02, Lot No. 1298684) dispensed at each 4 mL into another conical tube was transferred to a Ficoll tube as the blood-PBS mixture. The blood-PBS mixture was centrifuged at 840× g for 30 minutes at room temperature. PBMCs between Ficoll and the serum were collected, added with PBS and centrifuged at 210× g for 10 minutes. The supernatant was removed, and then a cell pellet was suspended in 3 mL of PBS and centrifuged at 210× g for 10 minutes. The supernatant was removed, and the cell pellet was added with 2 mL of RPMI (10% FBS+1% P/S). In the mixture, 10 μL was taken, the cell count was measured, and then 2×10⁵ cells/well was dispensed in a 96-well cell culture plate. 30 minutes after cell dispensing, 100 μL/well of each of recombinant PCV2b and PCV2d proteins were sensitized, incubated for 72 hours, and then centrifuged at 16,000× g for 10 minutes to separate the cell supernatant, and then stored at −70° C. until measured by ELISA.

### 2) Guinea Pig IFN-γ ELISA Assay

The amount of interferon gamma release was measured using the Guinea pig Interferon-65 (IFN-γ) ELISA Kit (Cusabio, Cat No. CSB-E06763Gu, Lot No. A13221097) according to the manufacturer's protocol. 100 μL/well of the standard and each serum sample were added to the enclosed plate, respectively, and the solution was removed from each well after incubation at 37° C. for 2 hours. Biotin-antibody (1×) was dispensed at each 100 μL/well and incubated at 37° C. for 1 hour. The plate was washed with 1X Wash Buffer three times at each 300 μL/well, added with HRP-avidin (1×) at each 100 μL/well and incubated at 37° C. for 1 hour. The plate was washed with 1X Wash Buffer 5 times at each 300 μL/well, and then dispensed with TMB substrate at each 90 μL/well and incubated at 37° C. for 15 to 30 minutes while blocking light. After adding each 50 μL/well of a stop solution, the absorbance (OD) value was measured at 450 nm using an Epoch™ Microplate Spectrophotometer (BioTek, USA).

### Experimental Example 3. PCV2b&d-ConCP Vaccine Efficacy Evaluation Test in Pigs

### 3-1. Design of Test

7-week-old pigs were used as a test subject, and test groups and vaccine antigen concentrations of vaccine used in the test were designed as shown in Table 4. The pig vaccination dose (1 dose) and the vaccination route (intramuscular injection) were set with reference to the standards of National Lot Release test. An additional challenge test was conducted at 13 weeks of age, and the challenge doses of a test group and challenge strains used in this test are shown in Table 5. The test schedule was performed as shown in Table 6.

TABLE 4

| | | Vaccine antigen | Vaccination | Subject | Vaccination |
|---|---|---|---|---|---|
| Group No. | Group name | concentration | dose | number | route |
| G4 | PCV2b& | 10 μg/2 mL/dose | 2 mL | 5 | Intramuscular |
| | d-ConCP | | | | vaccination |
| G5 | CircoFlex | — | 1 mL | 5 | Intramuscular |
| | | | | | vaccination |
| G6 | Challenge | — | 2 mL | 4 | Intramuscular |
| | control (PBS) | | | | vaccination |
| G7 | Non-challenge | — | 2 mL | 3 | Intramuscular |
| | control (PBS) | | | | vaccination |

TABLE 5

| Group | | Challenge dose | | Subject | |
|---|---|---|---|---|---|
| No. | Group name | TCID₅₀/mL | mL | number | Challenge route |
| G4 | PCV2b& | 5.5 | 2 mL | 5 | Nasal cavity |
| | d-ConCP | | | | |
| G5 | CircoFlex | 5.5 | 2 mL | 5 | Nasal cavity |
| G6 | Challenge | 5.5 | 2 mL | 4 | Nasal cavity and |
| | control (PBS) | | | | respiratory tract |
| G7 | Non-challenge | | | 3 | |
| | contro l(PBS) | | | | |

TABLE 6

| | Days | Days | |
|---|---|---|---|
| Weeks | postVaccination | postChallenge | Performance details |
| 3 weeks of age | — | — | Body weight measurement and blood |
| | | | collection |
| 4 weeks of age | — | — | Body weight measurement and blood |
| | | | collection |
| 5 weeks of age | — | — | Body weight measurement and blood |
| | | | collection |
| 6 weeks of age | — | — | Body weight measurement and blood |
| | | | collection |
| 7 weeks of age | — | — | Body weight measurement and blood |
| | | | collection |
| | | | Vaccination (DPV, Days post Vaccination) |
| 8 weeks of age | 7 | — | Body weight measurement |
| 9 weeks of age | 14 | — | Body weight measurement and blood |
| | | | collection |
| 10 weeks of | 21 | — | Body weight measurement |
| age | | | |
| 11 weeks of | 28 | — | Body weight measurement and blood |
| age | | | collection |
| 12 weeks of | 35 | — | Body weight measurement |
| age | | | |
| 13 weeks of | 42 | 0 | Body weight measurement and blood |
| | | | collection (PBMC) |
| age | | | Challenge (DPC, Days post Challenge) |
| 14 weeks of | 49 | 7 | Body weight measurement and blood |
| age | | | collection |
| 15 weeks of | 56 | 14 | Body weight measurement and blood |
| age | | | collection |
| 16 weeks of | 63 | 21 | Body weight measurement and blood |
| age | | | collection |
| | | | Post-mortem autopsy (Lung, Lymphnodes) |

### 3-2. Breeding Method of Pigs

To prevent infection in a test farm during a test period, 3-week-old pigs were bred in hoggery after 2 weeks of disinfection. All pigs freely consumed feed and drinking water during the test period. In 7-week-old pigs, in PCV2b&d-ConCP group (G4) and CircoFlex group (G5), the vaccine was vaccinated into the ear muscle (lateral neck muscle), and in challenge control group (G6) and non-challenge control group (G7), PBS was vaccinated under the same conditions. Six weeks after vaccination, 13-week-old PCV2b&d-ConCP group (G4) and CircoFlex group (G5) were challenged intranasally, and a challenge control group (G6) was challenged separately into the nasal cavity and respiratory tract. Three weeks after the challenge, samples were collected using swabs from the nasal cavity and rectum in all 16-week-old groups, and after euthanasia, the autopsy was performed and the lung lesions and enlarged inguinal lymph nodes were confirmed. Additionally, the lung and inguinal lymph node tissues were collected for tissue analysis and virus isolation. Body weights were measured every week during the test period, and blood was collected at two-week intervals after vaccination until the challenge date (13 weeks of age) and at one-week intervals after the challenge (13 weeks of age).

### 3-3. Quantification of Vaccine Antigen Concentration and Preparation and Vaccination of Vaccine

### 1) Vaccine Preparation and Vaccination

The concentration of the purified antigen was quantified through BCA Protein assay and then diluted in Phosphate Buffered Saline (PBS) so that the antigen concentration was 10 μg/mL/dose (1 dose of pig). MONTANIDE™ IMS1313 (Seppic, IMS1313 VG N) was used as an excipient and mixed with the antigen diluted in PBS at a ratio of 50:50 (v/v). Referring to the Standards of National Lot Release test, 2 mL of vaccine with a final antigen amount of 10 μg (1 dose of pig) was intramuscularly vaccinated into the ear muscle (lateral neck muscle) of 7-week-old pigs. A CircoFlex vaccination group was intramuscularly injected at each 1 mL according to a method and a dose.

### 3-4. PCV2 Challenge

1) Preparation of PCV2 for challenge

PK-15 cells were incubated in a T-75 cm² flask (SPL, Cat No.70075, LotNo.BBCE11A70075) using DMEM (GeneDEPOT, Cat No. CM002, Lot No.5292011) added with 8% FBS (GenDEPOT, Cat No. F0600-050, Lot No. 5101912) and prepared. The cells were vaccinated with a PCV2 strain (HID9071) and adsorbed at 37° C. for 1 hour, and then added with DMEM containing 1% Antibiotic-Antimycotic and incubated at 37° C. and 5% CO₂ conditions. After 24 hours, the virus culture medium was removed, and then the cells were washed three times using PBS, added with 300 mM Glucosamine, and incubated at 37° C. for 30 minutes. Glucosamine was removed, and the cells were washed three times using PBS, and then added with 20 mL of DMEM containing 1% Antibiotic-Antimycotic and incubated at 37° C. and 5% CO₂ for 3 days. The cells were harvested by repeating freezing and thawing three times, IFA was performed to measure the titers, and as a result, the titers were confirmed to be TCID₅₀/mL.

### 2) PCV2 Challenge

For challenge, at 13 weeks of age (42 DPV), a total of 2 mL of PCV2 was vaccinated into each nasal cavity of pigs, each 1 mL, in the PCV2b&d-ConCP group (G4), the CircoFlex group (G5), and the Challenge control group (G6) at a concentration of 10^{5.5}TCID₅₀/mL. In the Challenge control group (G6), two subjects G6-1 and G6-2 were challenged intratracheally.

### 3-5. Method for Analyzing PCV2b and PCV2d Antibody Levels in Pigs

### 1) Indirect ELISA

The purified PCV2b and PCV2d antigens were diluted in 0.05 M sodium bicarbonate buffer (pH 9.4) and dispensed at each 100 μL/well (100 ng) on a MaxiSorp 96-well plate and coated at 4° C. for 16 to 24 hours. The coating solution was removed and the plate was washed three times with 0.05% PBS-T at each 300 μL/well. 1% Bovine Serum Albumin (BSA) was diluted in PBS, dispensed at each 200 μL/well into each well, and blocked for 2 hours at 37° C. The blocking solution was removed and the plate was washed three times with 0.05% PBS-T at each 300 μL/well. For the primary antibody, a pig serum sample was diluted at 1:2,000 and reacted at 37° C. for 2 hours. The primary antibody was removed and the plate was washed three times with 0.05% PBS-T at each 300 μL/well. As a secondary antibody, Goat anti-Pig IgG FC-Fragment HRP Conjugate (Bethyl, Cat No. A100-104P) or Goat pAb to Pig IgA(HRP) (abcam, Cat No. ab112746, Lot No. GR272913-10) diluted at 1:10,000 was added at each 100 μL/well and then reacted at 37° C. for 1 hour. The secondary antibody was removed and the plate was washed three times with 0.05% PBS-T at each 300 μL/well. As a substrate, TetraMethyl-Benzidine (TMB, Surmodics, USA, Cat No. TMBW-1000-01, Lot No.TMBWW18) was added at each 100 μL/well, reacted in the dark for 5 minutes at room temperature, and then added with 2 N H₂SO₄ at each 100 μL/well, and then the reaction was stopped. The absorbance (OD) value was measured at a wavelength of 450 nm using an Epoch™ Microplate Spectrophotometer (Bio Tek, USA).

### 2) Examination of Neutralizing Antibody Titers

Test serum was non-assimilated at 56° C. for 30 minutes. Using a 96-well plate, the serum was diluted two-fold, dispensed with an equal amount of PCV2b or PCV2d (400 TCID₅₀/mL), and reacted at 37° C. for 1 hour. PCV-1 free porcine kidney (PK-15) cells were dispensed at 2×10⁴ cells/100 μl/well and incubated in an incubator for 3 days, and then Immunofluorescence assay (IFA) was performed as follows. The culture medium was removed, and the cells were washed once with PBS, then the PBS was completely removed and the plate was dried well. The dried plate was dispensed with each 100 μL/well of 80% cold acetone and then fixed at 4° C. for 1 hour (or −20° C. for 15 minutes). The fixing solution was removed and the plate was washed once with PBS, and then Pig anti-PCV2 antiserum was diluted at 1:500, dispensed at each 100 μL/well, and reacted at 37° C. for 1 hour. After washing three times with PBS, Goat anti-Pig IgG h+I FITC Conjugated (Bethyl, Cat No. A100-105F, Lot No. A100-105F-20) was diluted at 1:200, dispensed at each 100 μL/well, and reacted at 37° C. for 30 minutes. After washing three times with PBS, the plate was observed under a fluorescence microscope, and the reciprocal of the highest serum dilution that inhibited PCV2 proliferation was determined as neutralizing antibody titers.

### 3-6. Examination of Interferon Gamma (IFN-γ) Release Amount in Pig PBMCs

### 1) Preparation of Samples for Measuring Cytokine Release Amount (PBMC Separation)

4 mL of blood and 4 mL of PBS were mixed in a conical tube, and then Ficoll (GE Healthcare, Cat No. 17-1440-02, Lot No. 1298684) dispensed at each 4 mL into another conical tube was transferred to a Ficoll tube as the blood-PBS mixture. The blood-PBS mixture was centrifuged at 840× g for 30 minutes at room temperature. PBMCs between Ficoll and the serum were collected, added with PBS and centrifuged at 210× g for 10 minutes. The supernatant was removed, and then a cell pellet was suspended in 3 ml of PBS and centrifuged at 210× g for 10 minutes. The supernatant was removed, and the cell pellet was added with 2 mL of RPMI (10% FBS+1% P/S). In the mixture, 10 μL was taken, the cell count was measured, and then 2×10⁵ cells/well was dispensed in a 96-well cell culture plate. 30 minutes after cell dispensing, 100 μL/well of each of recombinant PCV2b and PCV2d proteins were sensitized, incubated for 72 hours, and then centrifuged at 16,000× g for 10 minutes to separate the cell supernatant, and then stored at −70° C. until measured by ELISA.

### 2) Pig Interferon-γ (IFN-γ) ELISA Assay

The amount of interferon gamma release was measured using an IFN-γ Porcine ELISA Kit (Invitrogen, Cat No. KSC4021, Lot No. 224978-007) according to the manufacturer's protocol. The enclosed plate was added with standards at each 100 μL/well. The remaining wells except for the standard were added with 50 μL/well each of a standard diluent buffer and added with 50 μL of a PBMC cell culture medium. All the wells except for a Chromogen blank well were added with 50 μL of a SW IFN-γ Biotin Conjugate solution, covered with a plate cover, and reacted at 37° C. for 2 hours. The wells were washed 4 times with a 1X wash buffer at each 300 μL/well. All the wells except for a Chromogen blank well were added with a 1X Streptavidin-HRP solution at each 100 μL/well, covered with a plate cover, and reacted at 37° C. for 30 minutes. The wells were washed 4 times with a 1X wash buffer at each 300 μL/well. The wells were added with Stabilized Chromogen at each 100 μL/well, reacted in the dark at room temperature for 30 minutes, and then added with a stop solution at each 100 μL/well to stop the reaction. The absorbance (OD) value was measured at a wavelength of 450 nm using an Epoch™ Microplate Spectrophotometer (BioTek, USA).

### 3-7. Viral DNA Measurement in Pig Blood and Tissue

### 1) Measurement of PCV2d Viremia in Pig Blood

Blood was collected at 2-week intervals after vaccination (0 DPV), and the blood was collected at 14 days (14 DPV), 28 days (28 DPV), and 42 days (42 DPV) after vaccination. After challenge (42 DPV), blood was collected every week, and the blood was collected at 7 days (49 DPV), 14 days (56 DPV), and 21 days (63 DPV) after the challenge, and serum was isolated. Viral DNA in serum was isolated using a Ribospin™ vRDplus Kit (GeneAll, Cat No. 312-150, Lot No. 31221a08001) according to the manufacturer's protocol, and RT-qPCR was performed by SYBR Green I (Enzynomics, Cat No. RT501M, Lot No. KE) to confirm the PCV2 DNA copy number.

### 2) Measurement of PCV2 DNA Loads in Pig Tissues (Lung and Lymph Nodes)

On day 21 (63DPV) after challenge, all groups were euthanized, and lung tissue and lymph nodes were collected for each subject. Tissues and PBS were added to a 2 mL E-tube at a ratio of 1:1 (g/mL), and for homogenization, steal beads were added and Tissue lyser II (Quiagen, Sesrial No.128140260) was used, and the homogenization was performed 3 times for 5 minutes at 2×24 Adapter (Quiagen, Cat No. 69982, Lot No. 130163660) 30 Hz, and further performed depending on a tissue homogeneous state. Thereafter, the mixture was centrifuged at 10,000× g for 5 minutes to isolate the supernatant. Viral DNA was isolated using a Ribospin™ vRDplus Kit (GeneAll, Cat No. 312-150, Lot No. 31221a08001) according to the manufacturer's protocol, and RT-qPCR was performed by SYBR GreenI(Enzynomics, Cat No. RT501M, Lot No. KE) to confirm the PCV2 DNA copy number.

### 3) Measurement of PCV2d DNA Loads in Pig Nasal and Rectal Swab

On day 21 (63 DPV) after challenge, all groups were euthanized, and samples were collected from the nasal cavity and rectum for each subject using a cotton swab. 1 mL of PBS and the swab samples were voltexed in a 2 mL E-tube, and centrifuged at 10,000× g for 5 minutes to separate the supernatant. Viral DNA was isolated using a Ribospin™ vRDplus Kit (GeneAll, Cat No. 312-150, Lot No. 31221a08001) according to the manufacturer's protocol, and RT-qPCR was performed by SYBR GreenI(Enzynomics, Cat No. RT501M, Lot No. KE) to confirm the PCV2 DNA copy number.

### 3-8. Pathological Evaluation

### 1) Confirmation of Gross Lesions

On day 21 (63 DPV) after challenge, all groups were euthanized, and autopsied for each subject, and gross lesions were confirmed in lung tissue and lymph nodes.

### 2) Immunohistochemistry (IHC)

Lung and lymphatic tissues were fixed in 10% neutral formalin and microtomy was performed. Histological evaluation of the lungs showed localized multifocal inflammatory lesions including bronchus-associated lymphatic tissue (hereinafter, BALT), inflammatory cell (monocyte) infiltration around blood vessels and bronchus (bronchioles), and interstitial pneumonia associated with diffuse alveolar wall thickening. BALT was graded semiquantitatively according to a frequency of observation into a normal level (often, 0), a slight increase (quite often, 1), a frequent increase (frequent, 2), and a significant increase (very frequent, 3), and for a more quantitative comparison, the number of lymphatic nodules with distinct nodular shapes was measured in two tissue sections per subject and the results between groups were compared. In the case of inflammatory lesions and diffuse interstitial pneumonia, grading was performed into normal range (0), mild (1), moderate (2), and severe (3), respectively. In the case of lymph nodes, if there were lesions, the details were recorded and the grading was performed into normal range (0), mild (1), moderate (2), and severe (3) using a semi-quantitative method, respectively. In order to compare the activation of a B cell region, the number of secondary lymphatic nodules was counted at a microscope magnification of 400× and the results between groups were compared. Tissue sections were prepared in two different areas from the lung and lymphatic tissues, and embedded in paraffin through a general tissue processing process. These tissues were micro-sectioned into 3 cm-thick sections, and then stained with hematoxylin and eosin, and examined using an optical microscope. Immunostaining for PCV2 was performed using a VECTASASTAIN® Elite® ABC Universal Kit (Vector Laboratories, Cat No. ZF0924). The paraffin-embedded lung and lymphatic tissues were attached to a special slide for immunostaining and then deparaffinized and hydrated. For antigen exposure (antigen unmasking), an Antigen Unmasking Solution (citrate-based, pH 6.0) (VECTOR Laboratories, Cat No. H-3300) was preheated in the microwave for 7 minutes, added with the tissue, and then additionally heated for 12 minutes. The Antigen Unmasking Solution was left to cool sufficiently, washed with running tap water, further washed with distilled water, and then treated in a solution containing methanol and 0.3% hydrogen peroxide for 30 minutes to inactivate endogenous peroxidase. After washing with PBS several times, the tissue was incubated with diluted normal horse blocking serum for 30 minutes at room temperature. After shaking off the normal blocking serum, a primary antibody Rabbit polyclonal anti-porcine PCV2 antibody (Invitrogen, Cat No. PAS-34969) diluted 1:500 was dispensed and left overnight at 4° C. In the case of a negative control, PBS was applied instead of the primary antibody. After washing with PBS. a diluted biotin-attached secondary antibody was dispensed and reacted at room temperature for 40 minutes. After washing with PBS, the secondary antibody reacted with a VECTASASTAIN® Elite® ABC reagent at room temperature for 30 minutes and then developed with a peroxidase substrate (DAB) for about 1 minute. After counter staining with hematoxylin, the secondary antibody was dehydrated and purified, encapsulated in cover glass, and positive or not was confirmed using an optical microscope.

### 3-9. Analysis of Body Weight Changes by Week and Average Daily Weight Gain (Hereinafter ADWG)

Body weights were measured for pigs in all groups by placing one pig in a cage installed on a scale. Using the measured body weights, the average daily weight gain (ADWG) for each subject was analyzed.

### Experimental Example 4. Statistical Analysis

Statistical analysis was performed using GraphPad Prism software. One-way ANOVA was used for comparison between groups, and in a post-hoc test, significant differences were confirmed using a Dunnett's multiple comparisons test. Based on a control group, in the case of P<0.05, it was determined to be statistically significant.

### EXAMPLES

### Example 1. PCV2b&d-ConCP Vaccine Efficacy Evaluation Test in Guinea Pigs

### 1-1. Pierce BCA Protein Assay Results for Quantification of Vaccine Antigen Concentrations

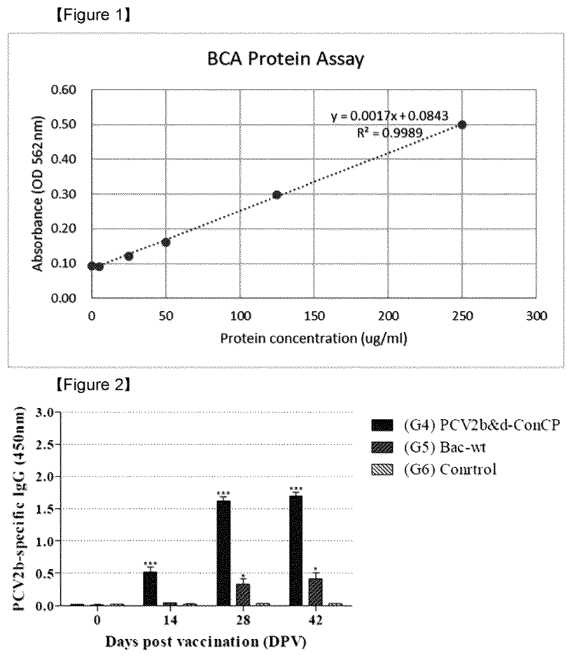

The concentration of vaccine antigen for the antigen purified for vaccination was quantified according to a method in the Pierce BCA Protein Assay kit (ThermoFisher, Cat No. 23227, Lot No. VA294776D), and an R-squared (coefficient of determination) value was confirmed as 0.9989 through the standard curve (working range=5 to 250 μg/mL). Quantitative results were shown in FIG. 1 and Tables 7 and 8.

TABLE 7

| | | | Average |
|---|---|---|---|
| | | OD value | OD value |
| Label | μg/mL | (562 nm) | (562 nm) |
| Standard A | 250 | 0.499 | 0.50 |
| | | 0.502 | |
| Standard B | 125 | 0.301 | 0.30 |
| | | 0.295 | |
| Standard C | 50 | 0.162 | 0.16 |
| | | 0.162 | |
| Standard D | 25 | 0.124 | 0.12 |
| | | 0.319 | |
| Standard E | 5 | 0.092 | 0.09 |
| | | 0.09 | |
| Standard F | 0 | 0.091 | 0.09 |

TABLE 8

| | | Quantitative |
|---|---|---|
| | OD value | value |
| Label | (562 nm) | (μg/ml) |
| PCV2b&d-ConCP | 0.115 | 18 |
| Bac-wt | 0.281 | 116 |

As shown in FIG. 1 and Tables 7 and 8, the quantitative value of PCV2b&d ConCP was a total of 1,080 μg based on 60 mL of ConCP antigen purified to 18 μg/mL, and the quantitative value of Bac-wt was 116 μg/mL, and a total of 1,160 μg of antigen was prepared based on 10 mL of the purified wt antigen. Thereafter, in guinea pig and pig tests, the vaccine was diluted according to the vaccine antigen concentration and injected.

1-2. PCV2b-specific IgG Antibody Titer and PCV2d-specific IgG Antibody Titer Tests in Guinea Pigs

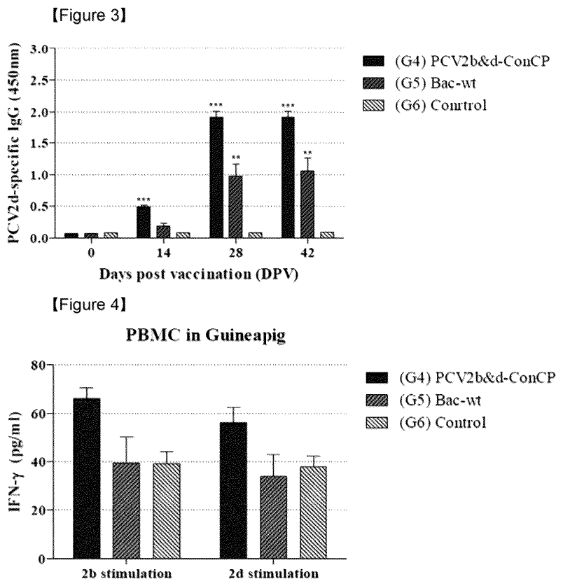

In order to evaluate the efficacy of the PCV2b&d-ConCP vaccine in guinea pigs, serum was isolated after vaccination for each group, and PCV2b or PCV2d antigen-specific IgG antibody levels were confirmed by indirect ELISA. The PCV2b-specific IgG antibody levels test results were shown in FIG. 2 and Table 9, and the PCV2d-specific IgG antibody levels were shown in FIG. 3 and Table 10.

TABLE 9

| | | PCV2b-specific IgG antibody levels in guinea pigs | | | |
|---|---|---|---|---|---|
| Group | | (OD450, mean ± standard error) | | | |
| No. | Group name | 0 DPV | 14 DPV | 28 DPV | 42 DPV |
| G4 | PCV2b&d-ConCP | 0.02 ± 0.00 | 0.51 ± 0.08 | 1.62 ± 0.06 | 1.69 ± 0.06 |
| G5 | Bac-wt | 0.01 ± 0.00 | 0.04 ± 0.01 | 0.33 ± 0.08 | 0.41 ± 0.30 |
| G6 | Control (PBS) | 0.02 ± 0.00 | 0.02 ± 0.00 | 0.04 ± 0.00 | 0.03 ± 0.00 |

TABLE 10

| | | PCV2d-specific IgG antibody levels in guinea pigs | | | |
|---|---|---|---|---|---|
| Group | | (OD450, mean ± standard error) | | | |
| No. | Group name | 0 DPV | 14 DPV | 28 DPV | 42 DPV |
| G4 | PCV2b&d-ConCP | 0.07 ± 0.00 | 0.48 ± 0.04 | 1.91 ± 0.30 | 1.92 ± 0.09 |
| G5 | Bac-wt | 0.08 ± 0.00 | 0.18 ± 0.05 | 0.97 ± 0.20 | 1.06 ± 0.20 |
| G6 | Control (PBS) | 0.09 ± 0.01 | 0.09 ± 0.00 | 0.09 ± 0.00 | 0.09 ± 0.00 |

As a test result, it was confirmed that a Control group (G6) vaccinated with PBS showed low IgG antibody levels in all test periods, but a PCV2b&d-ConCP group (G4) vaccinated with the developed vaccine induced the production of PCV2b or PCV2d antigen specific IgG antibody from 14 DPV. When compared to the control group, there was a very significant difference. In addition, a relatively low level of IgG antibody production was induced even in a Bac-wt vaccinated group (G5), but there was a significant difference compared to the control group.

PBMCs were isolated from guinea pig blood at 42 days after group-specific vaccination (½ dose of pig vaccination) in guinea pigs, and the amount of interferon gamma release was measured using a Guinea pig Interferon-γ (IFN-γ) ELISA Kit (Cusabio, Cat No. CSB-E06763Gu, Lot No. A13221097). The results of measuring the amount of interferon gamma release were shown in FIG. 4.

As shown in FIG. 4, the Control group (G6) injected with PBS and the Bac-wt vaccinated group (G5) showed similar levels of interferon gamma release amount. Meanwhile, the PCV2b&d-ConCP group (G4) vaccinated with the developed vaccine showed the highest release amount with an average of 66 pg/mL for PCV2b antigen stimulation and 56.25 pg/mL for PCV2d antigen stimulation.

### 1-3. Results of Examining Neutralizing Antibody Titers Against PCV2b and PCV2d in Guinea Pigs

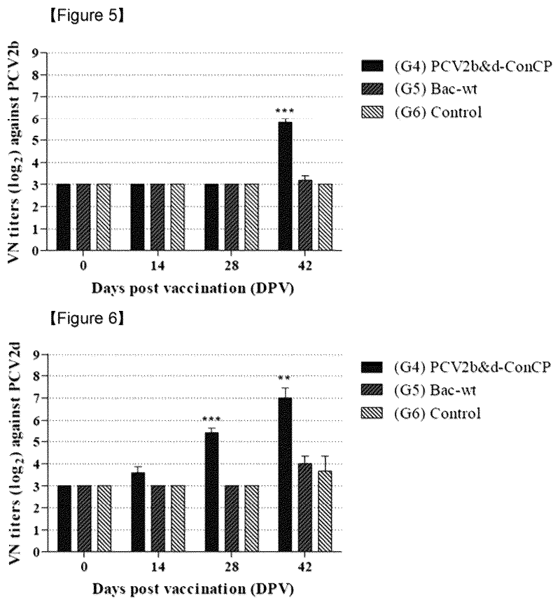

A test was conducted to confirm neutralizing antibody titers against PCV2b and PCV2d of the PCV2b&d-ConCP vaccine in guinea pigs. The results of confirming neutralizing antibody titers against PCV2b were shown in FIG. 5 and Table 11, and the results of confirming neutralizing antibody titers against PCV2d were shown in FIG. 6 and Table 12.

TABLE 11

| | | Neutralizing antibody titers against PCV2b in guinea pigs | | | |
|---|---|---|---|---|---|
| Group | | (mean ± standard error) | | | |
| No. | Group name | 0 DPV | 14 DPV | 28 DPV | 42 DPV |
| G4 | PCV2b&d-ConCP | 8.0 ± 0.0 | 8.0 ± 0.0 | 8.0 ± 0.0 | 57.6 ± 6.4 |
| G5 | Bac-wt | 8.0 ± 0.0 | 8.0 ± 0.0 | 8.0 ± 0.0 | 9.6 ± 1.6 |
| G6 | Control(PBS) | 8.0 ± 0.0 | 8.0 ± 0.0 | 8.0 ± 0.0 | 8.0 ± 0.0 |

TABLE 12

| | | Neutralizing antibody titers against PCV2d in guinea pigs | | | |
|---|---|---|---|---|---|
| Group | | (mean ± standard error) | | | |
| No. | Group name | 0 DPV | 14 DPV | 28 DPV | 42 DPV |
| G4 | PCV2b&d-ConCP | 8.0 ± 0.0 | 12.8 ± 2.0 | 44.8 ± 7.8 | 153.6 ± 43.4 |
| G5 | Bac-wt | 8.0 ± 0.0 | 8.0 ± 0.0 | 8.0 ± 0.0 | 17.6 ± 3.9 |
| G6 | Control (PBS) | 8.0 ± 0.0 | 8.0 ± 0.0 | 8.0 ± 0.0 | 16.0 ± 8.0 |

As shown in FIG. 5 and Table 11, it was confirmed that up to 28 DPV after vaccination, in the Control group (G6) injected with PBS and the Bac-wt vaccinated group (G5), low neutralizing antibody titers of average 16 times or less were shown in all test periods, but in the PCV2b&d-ConCP group (G4) vaccinated with the developed vaccine, the production of neutralizing antibody titers of 32 times or more was induced from 42 DPV. In particular, it was confirmed that when compared to the Control group (G6), the largest difference was shown at 42 DPV for PCV2b, and a very significant difference was shown by inducing higher levels of neutralizing antibody titers at an average of 7.2 times. As shown in FIG. 6 and Table 12, it was confirmed that up to 28 DPV after vaccination, in the Control group (G6) vaccinated with PBS and the Bac-wt vaccinated group (G5), low neutralizing antibody titers of average 16 times or less were shown in all test periods, but in the PCV2b&d-ConCP group (G4) vaccinated with the developed vaccine, the production of neutralizing antibody of 32 times or more was induced from 28 DPV. In particular, it was confirmed that when compared to the Control group (G6), the largest difference was shown at 42 DPV for PCV2d, and a very significant difference was shown by inducing higher levels of neutralizing antibody titers at an average of 19.2 times.

### Example 2. PCV2b&d-ConCP Vaccine Efficacy Evaluation Test in Pigs

### 2-1. Results of Measuring Antibody Titers

1) Measurement of PCV2b-specific IgG Antibody levels and PCV2d-Specific IgG Antibody Levels in Pigs

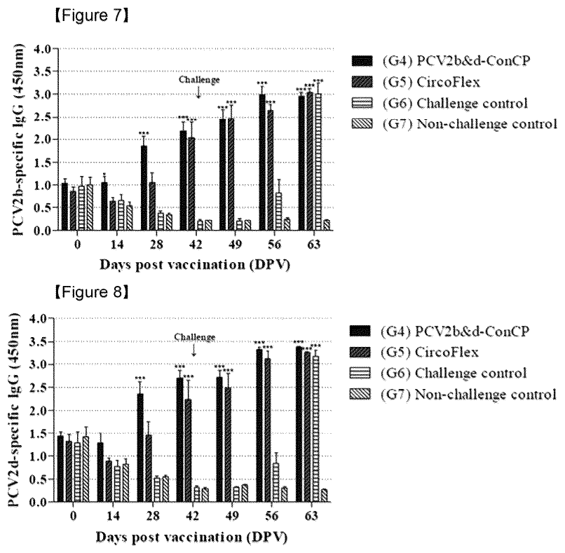

In order to evaluate the efficacy of the PCV2b&d-ConCP vaccine in pigs as a target animal, serum was isolated after vaccination for each group, and PCV2b or PCV2d antigen-specific IgG antibody levels were confirmed by indirect ELISA. The results of measuring the PCV2b-specific IgG antibody levels were shown in FIG. 7 and Table 13, and the results of measuring the PCV2d-specific IgG antibody levels were shown in FIG. 8 and Table 14.

TABLE 13

| | | PCV2b-specific IgG antibody levels in pigs | | | | | | |
|---|---|---|---|---|---|---|---|---|
| | | (OD450, mean ± standard error) | | | | | | |
| Group | Group | | 14 | 28 | 42 | 49 | 56 | 63 |
| No. | name | 0 DPV | DPV | DPV | DPV | DPV | DPV | DPV |
| G4 | PCV2b&d- | 1.04 ± | 1.05 ± | 1.85 ± | 2.17 ± | 2.45 ± | 2.99 ± | 2.96 ± |
| | ConCP | 0.10 | 0.14 | 0.22 | 0.20 | 0.20 | 0.19 | 0.08 |
| G5 | CircoPlex | 0.85 ± | 0.62 ± | 1.04 ± | 2.02 ± | 2.46 ± | 2.65 ± | 3.04 ± |
| | | 0.10 | 0.10 | 0.22 | 0.34 | 0.31 | 0.13 | 0.09 |
| G6 | Challenge | 0.97 ± | 0.65 ± | 0.36 ± | 0.20 ± | 0.21 ± | 0.82 ± | 3.01 ± |
| | control | 0.22 | 0.13 | 0.06 | 0.03 | 0.03 | 0.29 | 0.23 |
| G7 | Non- | 0.99 ± | 0.53 ± | 0.33 ± | 0.21 ± | 0.21 ± | 0.24 ± | 0.21 ± |
| | challenge | 0.18 | 0.08 | 0.04 | 0.01 | 0.01 | 0.03 | 0.02 |
| | control | | | | | | | |

TABLE 14

| | | PCV2d-specific IgG sunibody levels in pigs | | | | | | |
|---|---|---|---|---|---|---|---|---|
| | | (OD450, mean ± standard error) | | | | | | |
| Group | Group | | 14 | 28 | 42 | 49 | 56 | 63 |
| No. | name | 0 DPV | DPV | DPV | DPV | DPV | DPV | DPV |
| G4 | PCV2b&d- | 1.44 ± | 1.29 ± | 2.35 ± | 2.69 ± | 2.71 ± | 3.33 ± | 3.37 ± |
| | ConCP | 0.10 | 0.22 | 0.26 | 0.17 | 0.15 | 0.05 | 0.02 |
| G5 | CircoFlex | 1.32 ± | 0.89 ± | 1.45 ± | 2.23 ± | 2.49 ± | 3.13 ± | 3.26 ± |
| | | 0.15 | 0.06 | 0.30 | 0.41 | 0.30 | 8.17 | 0.02 |
| G6 | Challenge | 1.29 ± | 0.77 ± | 0.51 ± | 0.32 ± | 0.32 ± | 0.83 ± | 3.19 ± |
| | control | 0.25 | 0.13 | 0.07 | 0.03 | 0.02 | 0.24 | 0.13 |
| G7 | Non- | 1.41 ± | 0.82 ± | 0.55 ± | 0.29 ± | 0.37 ± | 0.31 ± | 0.28 ± |
| | challenge | 0.21 | 0.13 | 0.05 | 0.03 | 0.01 | 0.03 | 0.02 |
| | control | | | | | | | |

As a result of examining PCV2b or PCV2d antigen-specific IgG antibody levels in pigs, all groups had maternal antibodies even before vaccination and thus showed relatively high levels of antibody. It was confirmed that in the Challenge control group (G6) and the Non-challenge control group (G7) vaccinated with PBS, maternal antibody levels began to decline from 14 DPV, but in the PCV2b&d-ConCP group (G4) vaccinated with the developed vaccine, high levels of IgG antibody were induced from 28 DPV. In particular, relatively higher levels of antibody were induced than a CircoFlex group (G5) vaccinated with a commercial vaccine, and there was a very significant difference compared to the Non-challenge control group (G7). In addition, after challenge (42 DPV), the vaccinated group maintained very high levels of antibody until the end of the defense evaluation test. As a result of comparing antibody levels against a PCV2 genotype, PCV2d antibody levels were approximately 1.13 times higher than PCV2b at the test end date (63 DPV).

### 2) Measurement of PCV2b-Specific IgA Antibody Levels and PCV2d-Specific IgA Antibody Levels in Pigs

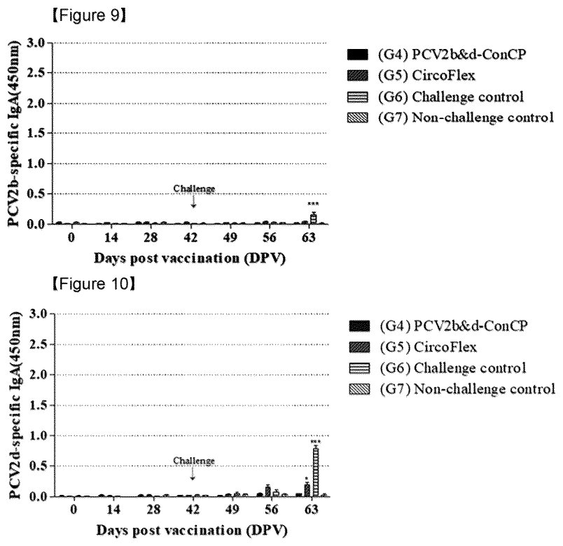

In order to evaluate the efficacy of the PCV2b&d-ConCP vaccine in pigs as a target animal, serum was isolated after vaccination for each group, and PCV2b or PCV2d antigen-specific IgA antibody levels were confirmed by indirect ELISA. The results of measuring the PCV2b-specific IgA antibody levels were shown in FIG. 9 and Table 15, and the results of measuring the PCV2d-specific IgA antibody levels were shown in FIG. 10 and Table 16.

TABLE 15

| | | PCV2b-specific IgA antibody levels in pigs | | | | | | |
|---|---|---|---|---|---|---|---|---|
| | | (OD450, mean ± standard error) | | | | | | |
| Group | Group | | 14 | 28 | 42 | 49 | 56 | 63 |
| No. | name | 0 DPV | DPV | DPV | DPV | DPV | DPV | DPV |
| G4 | PCV2b&d- | 0.08 ± | 0.08 ± | 0.10 ± | 0.11 ± | 0.09 ± | 0.10 ± | 0.10 ± |
| | ConCP | 0.01 | 0.01 | 0.01 | 0.01 | 0.01 | 0.03 | 0.01 |
| G5 | CircoFlex | 0.09 ± | 0.10 ± | 0.31 ± | 0.12 ± | 0.10 ± | 0.11 ± | 0.12 ± |
| | | 0.01 | 0.02 | 0.02 | 0.00 | 0.01 | 0.01 | 0.01 |
| G6 | Challenge | 0.06 ± | 0.08 ± | 0.08 ± | 0.12 ± | 0.10 ± | 0.10 ± | 0.25 ± |
| | control | 0.00 | 0.01 | 0.01 | 0.01 | 0.01 | 0.01 | 0.05 |
| G7 | Non- | 0.06 ± | 0.09 ± | 0.30 ± | 0.10 ± | 0.11 ± | 0.10 ± | 0.12 ± |
| | challenge | 0.00 | 0.01 | 0.03 | 0.01 | 0.03 | 0.01 | 0.03 |
| | control | | | | | | | |

TABLE 16

| | | PCV2d-specific IgA antibody levels in pigs | | | | | | |
|---|---|---|---|---|---|---|---|---|
| | | (OD450, mean ± standard error) | | | | | | |
| Group | Group | | 14 | 28 | 42 | 49 | 56 | 63 |
| No. | name | 0 DPV | DPV | DPV | DPV | DPV | DPV | DPV |
| G4 | PCV2b&d- | 0.11 ± | 0.12 ± | 0.19 ± | 0.12 ± | 0.16 ± | 0.26 ± | 0.22 ± |
| | ConCP | 0.01 | 0.02 | 0.03 | 0.01 | 0.02 | 0.02 | 0.01 |
| G5 | CircoFlex | 0.12 ± | 0.20 ± | 0.26 ± | 0.20 ± | 0.25 ± | 0.42 ± | 0.46 ± |
| | | 0.02 | 0.07 | 0.07 | 0.02 | 0.03 | 0.07 | 0.08 |
| G6 | Challenge | 0.08 ± | 0.12 ± | 0.11 ± | 0.14 ± | 0.17 ± | 0.24 ± | 1.23 ± |
| | control | 0.01 | 0.02 | 0.01 | 0.02 | 0.03 | 0.08 | 0.15 |
| G7 | Non- | 0.07 ± | 0.11 ± | 0.37 ± | 0.10 ± | 0.15 ± | 0.13 ± | 0.13 ± |
| | challenge | 0.01 | 0.01 | 0.02 | 0.01 | 0.03 | 0.01 | 0.02 |
| | control | | | | | | | |

As a result of examining PCV2b or PCV2d antigen-specific IgA antibody levels in pigs, the IgA antibody levels of all groups were significantly lower than those of IgG, but the Challenge control group (G6) induced relatively high levels of IgA antibody at 63 DPV, and showed a very significant difference when compared to the Non-challenge control group (G7). Unlike the vaccinated group, it was determined that the control groups were not vaccinated after the challenge and unable to neutralize PCV2d, so that IgA was produced during a process of forming defense immunity.

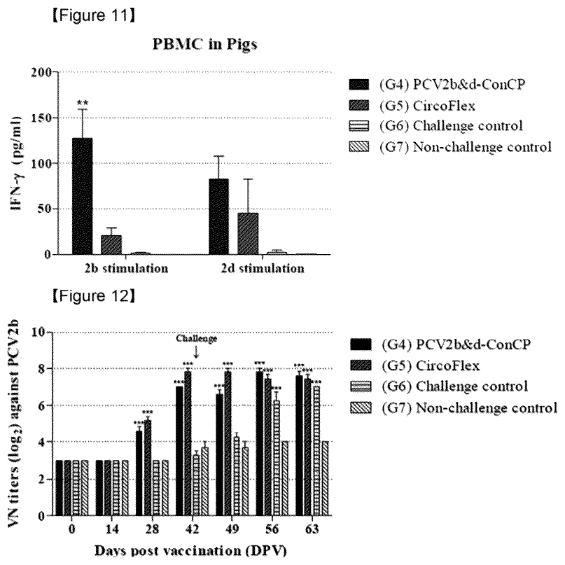

PBMCs were isolated from pig blood 42 days after vaccination in pigs, and the amount of interferon gamma release was measured using an Invitrogen IFN-γ Porcine ELISA Kit. The results of measuring the amount of interferon gamma release were shown in FIG. 11.

As shown in FIG. 11, the Challenge control group (G6) and Non-challenge control group (G7) vaccinated with PBS and the CircoFlex group (G5) vaccinated with the commercial vaccine had relatively low levels of interferon gamma release amount at an average of 50 pg/mL or less. Meanwhile, the PCV2b&d-ConCP group (G4) vaccinated with the developed vaccine showed the highest release amount with an average of 126.53 pg/mL for PCV2b antigen stimulation and 82.45 pg/mL for PCV2d antigen stimulation. In particular, the PCV2b stimulation results showed a very significant difference compared to the Non-challenge control group (G7).

### 2-2. Measurement of Neutralizing Antibody Titers Against PCV2b and PCV2d in Pigs

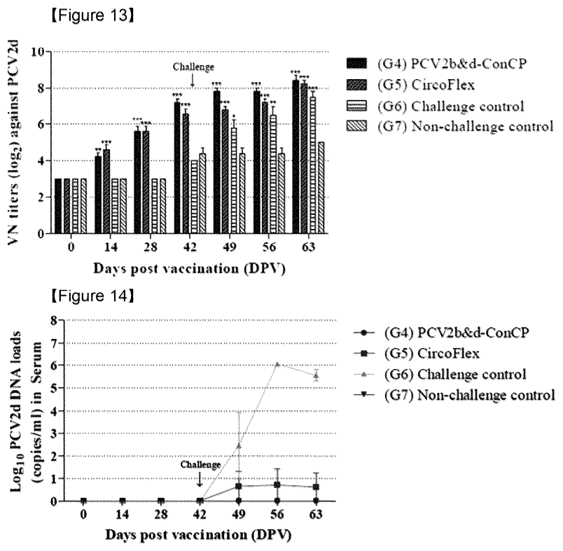

Neutralizing antibody titers against PCV2b and PCV2d of the PCV2b&d-ConCP vaccine in pigs were measured. The results of measuring neutralizing antibody titers against PCV2b were shown in FIG. 12 and Table 17, and the results of measuring neutralizing antibody titers against PCV2d were shown in FIG. 13 and Table 18.

TABLE 17

| | | Neutralizing antibody titers against PCV2b in pigs | | | | | | |
|---|---|---|---|---|---|---|---|---|
| | | (mean ± standard error) | | | | | | |
| Group | | | 14 | 28 | 42 | 49 | | 63 |
| No. | Group name | 0 DPV | DPV | DPV | DPV | DPV | 56 DPV | DPV |
| G4 | PCV2b&d- | 8.0 ± | 8.0 ± | 25.6 ± | 128.0 ± | 102.4 ± | 230.4 ± | 204.8 ± |
| | ConCP | 0.0 | 0.0 | 3.9 | 0.0 | 15.7 | 25.6 | 31.4 |
| G5 | CircoPlex | 8.0 ± | 8.0 ± | 38.4 ± | 230.4 ± | 230.4 ± | 179.2 ± | 179.2 ± |
| | | 0.0 | 0.0 | 6.4 | 25.6 | 25.6 | 31.4 | 31.4 |
| G6 | Challenge | 8.0 ± | 8.0 ± | 8.0 ± | 10.0 ± | 20.0 ± | 88.0 ± | 128.0 ± |
| | control | 0.0 | 0.0 | 0.0 | 2.0 | 4.0 | 24.0 | 0.0 |
| G7 | Non-challenge | 8.0 ± | 8.0 ± | 8.0 ± | 13.3 ± | 13.3 ± | 16.0 ± | 16.0 ± |
| | control | 0.0 | 0.0 | 0.0 | 2.7 | 2.7 | 0.0 | 0.0 |

TABLE 18

| | | Neutralizing antibody titers against PCV2d in pigs | | | | | | |
|---|---|---|---|---|---|---|---|---|
| | | (mean ± standard error) | | | | | | |
| Group | | | 14 | 28 | 42 | 49 | 56 | |
| No. | Group name | 0 DPV | DPV | DPV | DPV | DPV | DPV | 63 DPV |
| G4 | PCV2b&d- | 8.0 ± | 19.2 ± | 512 ± | 153.6 ± | 230.4 ± | 230.4 ± | 358.4 ± |
| | ConCP | 0.0 | 3.2 | 7.8 | 25.6 | 25.6 | 25.6 | 62.7 |
| G5 | CircoFlex | 8.0 ± | 25.6 ± | 51.2 ± | 102.4 ± | 115.2 ± | 153.6 ± | 307.2 ± |
| | | 0.0 | 3.9 | 7.8 | 15.7 | 12.8 | 25.6 | 51.2 |
| G6 | Challenge | 8.0 ± | 8.0 ± | 8.0 ± | 16.0 ± | 64.0 ± | 104.0 ± | 192.0 ± |
| | control | 0.0 | 0.0 | 0.0 | 0.0 | 22.6 | 24.0 | 37.0 |
| G7 | Non-challenge | 8.0 ± | 8.0 ± | 8.0 ± | 21.3 ± | 21.3 ± | 21.3 ± | 32.0 ± |
| | control | 0.0 | 0.0 | 0.0 | 5.3 | 5.3 | 5.3 | 0.0 |

As shown in FIG. 12 and Table 17, it was confirmed that neutralizing antibody titers against PCV2b showed low neutralizing antibody titers of average 16 or less in the Challenge control group (G6) and Non-challenge control group (G7) vaccinated with PBS up to 28 DPV after vaccination, but the PCV2b&d-ConCP group (G4) induced the production of neutralizing antibodies from 28 DPV. In particular, the PCV2b&d-ConCP group (G4) induced higher levels of neutralizing antibody titers against PCV2b of average 230.4, which was 51.2 higher than the CircoFlex group (179.2) based on 56 DPV, and there was a significant difference compared to the control group. As shown in FIG. 13 and Table 18, it was confirmed that neutralizing antibody titers against PCV2d showed low neutralizing antibody titers of average 16 or less in the Challenge control group (G6) and Non-challenge control group (G7) vaccinated with PBS up to 28 DPV after vaccination, but the PCV2b&d-ConCP group (G4) induced the production of neutralizing antibodies from 14 DPV. In particular, the PCV2b&d-ConCP group (G4) induced higher levels of neutralizing antibody titers against PCV2d of average 358.4, which was 51.2 higher than the CircoFlex group (307.2) based on 63 DPV, and there was a significant difference compared to the control group. In addition, neutralizing antibody titers against PCV2d were relatively high compared to PCV2b.

Therefore, through the results of confirming neutralizing antibody titers, the cross-neutralization reaction between PCV2b and PCV2d was confirmed, and it was confirmed that in pigs, the PCV2b&d-ConCP vaccine had more effective neutralizing antibody titers against PCV2b and PCV2d than a commercial CircoFlex vaccine.

### 2-3. Viral DNA Measurement in Pig Blood and Tissues

### 1) Measurement Results of PCV2d Viremia in Pig Blood

PCV2d viremia in pig blood by challenge after vaccination was analyzed, and the results were shown in FIG. 14.

As shown in FIG. 14, PCV2d viremia was not confirmed in the PCV2b&d-ConCP group (G4) after vaccination until the end of the test, but it was confirmed that in the Challenge control group (G6) vaccinated with PBS and the CircoFlex group (G5) vaccinated with the commercial vaccine, viremia was increased from 7 days (56 DPV) after challenge. In particular, the Challenge control group (G6) showed the highest level with an average of 1.21×10⁶ copies/mL based on 56 DPV, and had a very significant difference when compared to the Non-challenge control group (G7). Meanwhile, in the PCV2b&d-ConCP group (G4), like the Non-challenge control group (G7), PCV2d viremia was not confirmed even after challenge.

### 2) Measurement Results of PCV2d DNA Loads in Pig Lungs and Lymph Nodes

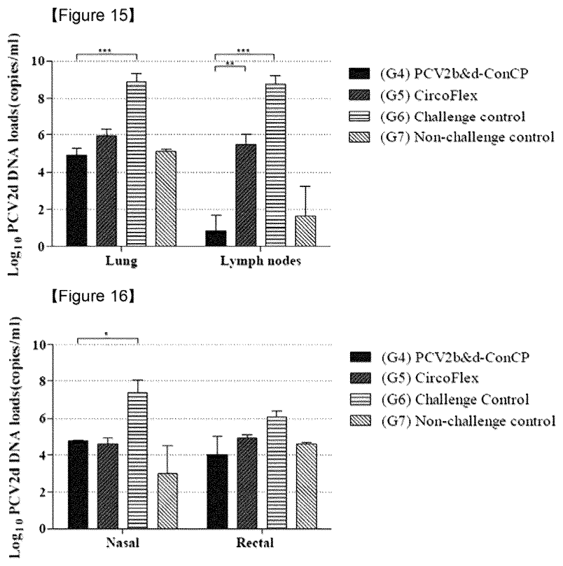

All test groups were euthanized at 63 DPV, and lung and lymph tissues were collected from each subject. Thereafter, viral DNA was extracted from each tissue sample and the amount of PCV2d DNA was measured through real time-qPCR. PCV2 DNA loads were measured in lungs and lymph nodes, and the results were shown in FIG. 15.

As shown in FIG. 15, the Challenge control group (G6) vaccinated with PBS had a significantly high level of PCV2d DNA copy number compared to other test groups, with average 2.37×10⁹ copies/mL in the lungs and average 1.78×10⁹ copies/mL in the lymph nodes. On the other hand, the PCV2b&d-ConCP group (G4) showed average 1.98×10⁵ copies/mL or less, which was similar to the Non-challenge control group (G7). In particular, more effective defense against PCV2d challenge was shown than the CircoFlex group (G5) vaccinated with the commercial vaccine in the lymph nodes, and showed a very significant difference compared to the PCV2b&d-ConCP group (G4).

### 3) Results of Measuring PCV2d DNA Loads in Pig Nasal and Rectal Swab

Viral DNA was extracted from nasal and rectal swab and the amount of PCV2d DNA was measured through real time-qPCR, and the results were shown in FIG. 16.

As shown in FIG. 16, the Challenge control group (G6) injected with PBS had a very high level of PCV2d DNA copy number, with average 1.22×10⁸ copies/mL in the nasal sample and average 2.03×10⁶ copies/mL in the rectal sample. The PCV2b&d-ConCP group (G4) vaccinated with the developed vaccine showed average 4.32×10⁴ copies/mL, which was similar to the CircoFlex group (G5) vaccinated with the commercial vaccine, and showed a significant difference compared to the Challenge control group (G6). On the other hand, the rectal swab sample showed a relatively low level and had no significant difference between groups.

### 2-4. Pathological Evaluation Results

### 1) Confirmation of Gross Lesions

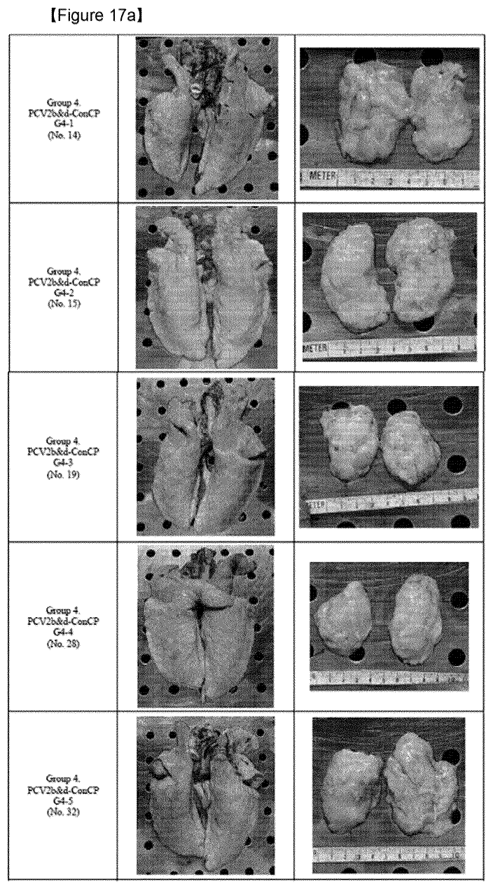

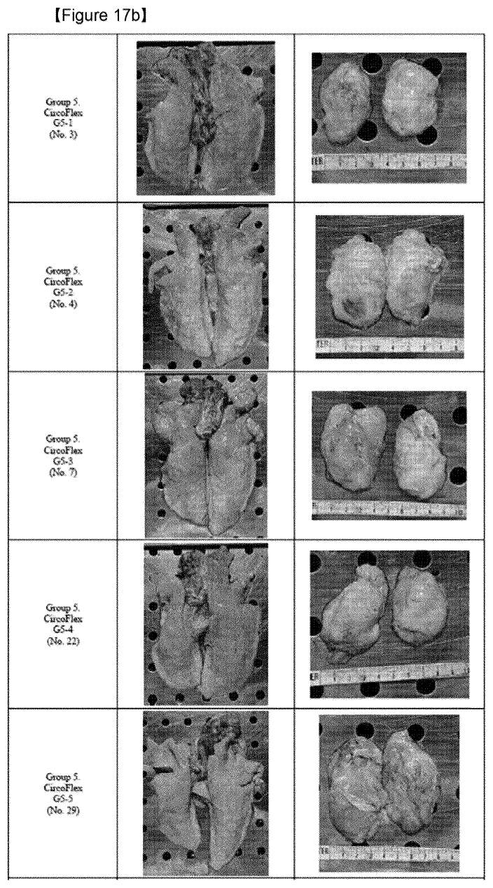

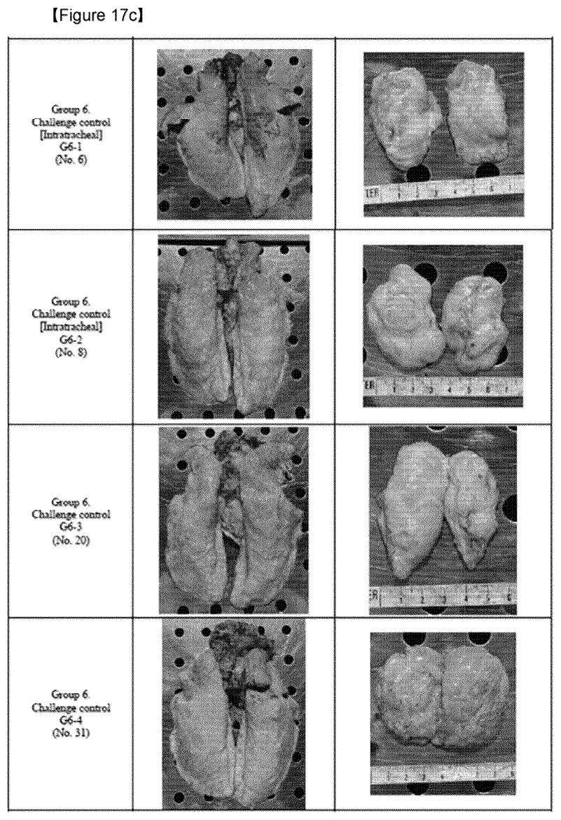

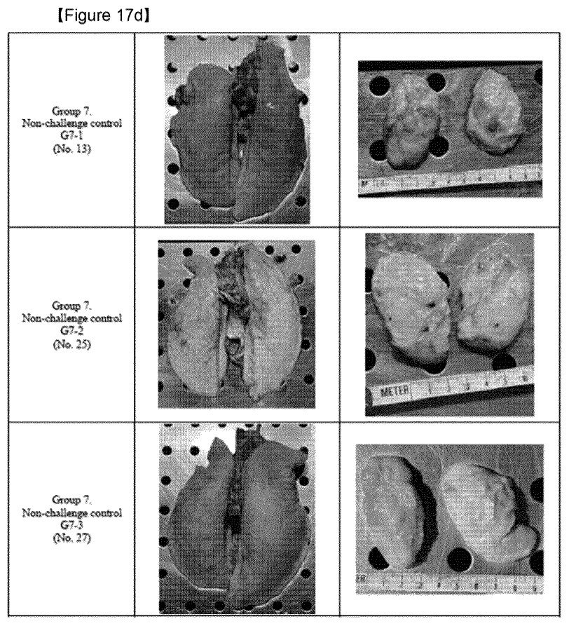

At the end of the experiment, 63 DPV, all groups were euthanized, and the lungs and lymph nodes of each subject were collected to confirm gross lesions. As shown in FIGS. 17A to 17D, there were no significant differences in lymphadenopathy and weight changes of lymph nodes in all test groups. However, compared with the Non-challenge control group (G7) vaccinated with PBS, some subjects in the Challenge control group (G6) and the CircoFlex group (G5) vaccinated with the commercial vaccine showed lesions similar to hemorrhage in the lungs and lymph nodes. On the other hand, no specific lesions were identified in the PCV2b&d-ConCP group (G4).

### 2) Histological Examination of Lungs

Findings observed in the pig lungs included proliferation of bronchus-associated lymphatic tissue, mononuclear inflammatory cell infiltrates mainly observed around the bronchi and blood vessels, interstitial pneumonia, etc.

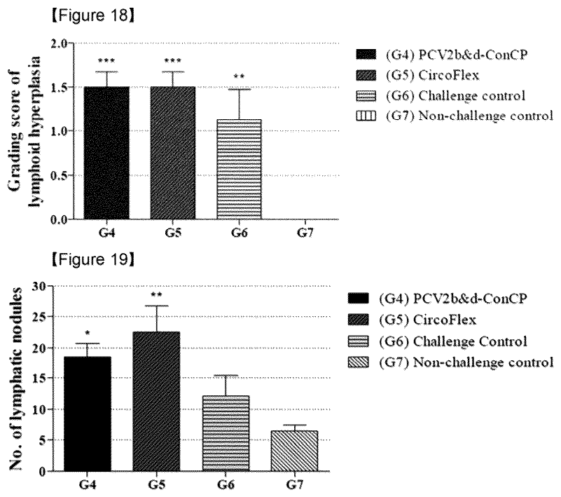

The proliferation of bronchus-associated lymphatic tissue (BALT) may increase in number and size when proliferation of lymphocytes is promoted by an infectious agent, and conversely, may decrease in number and size when the infectious agent causes lymphoid depletion. Therefore, in order to compare the effect on BALT in the test, semi-quantitative analysis was performed based on the frequency of BALT and the number of BALTs was counted, and the results were compared, and the results were shown in FIG. 18 and Table 20.

TABLE 20

| | G4 | | G6 | G7 |
|---|---|---|---|---|
| | (PCV2b& | G5 | (Challenge | (Non-challenge |
| Histopathology | d-ConCP) | (CircoFlex) | control) | control) |
| No. of animals (No. of | 5(10) | 5(10) | 4(8) | 3(6) |
| examined tissues) | | | | |
| Lymphoid hyperplasia, | 1.5 ± 0.53 | 1.5 ± 0.53 | 1.12 ± 0.99 | 0.0 ± 0.00 |
| BALT* | | | | |
| No. of lymphatic nodules | 18.5 ± 7.04 | 22.6 ± 12.9 | 12.1 ± 9.22 | 6.5 ± 2.35 |
| Inflammatory cell | 1.10 ± 0.32 | 1.2 ± 0.42 | 1.88 ± 0.83 | 1.83 ± 0.41 |
| infiltration, multifocal* | | | | |
| Interstitial pneumonia, | 1.10 ± 0.32 | 1.6 ± 0.52 | 2.1 ± 3.13 | 2.0 ± 0.89 |
| diffuse* | | | | |

*grading criteria; 1, minimal; 2, mild; 3, moderate; 4, severe

As illustrated in FIG. 18, as results of visual and semi-quantitative evaluation of the proliferation of bronchus-associated lymphatic tissue (BALT), the frequency of BALT was slightly increased in the Challenge control group (G6) compared to the Non-challenge control group (G7), and was higher on average in the PCV2b&d-ConCP group (G4) and the CircoFlex group (G5) than the Challenge control group (G6), but had no significant difference between the groups except for the Non-challenge control group (G7).

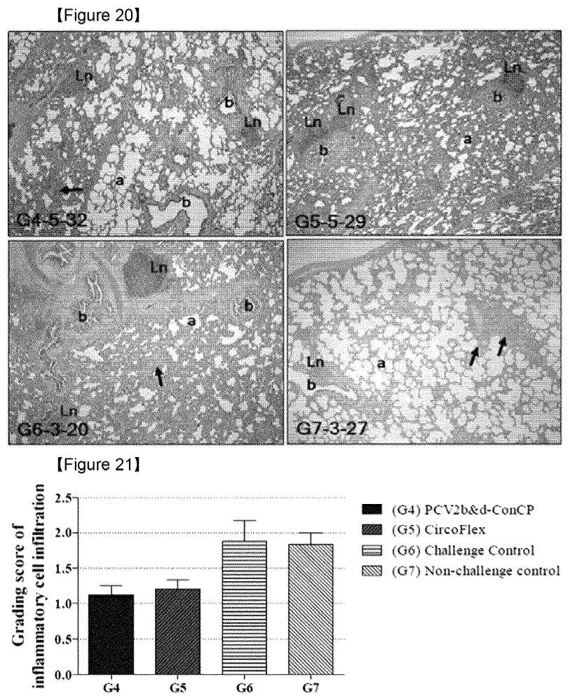

The number of lymphoid nodules around the bronchus was counted in two tissue sections for each subject, and as a result, as shown in FIGS. 19 and 20, the number of lymphoid nodules was increased in the Challenge control group (G6) compared to the Non-challenge control group (G7), but there was no significant difference. On the other hand, the number of lymphoid nodules was further increased in the PCV2b&d-ConCP group (G4) and the CircoFlex group (G5), and there was a significant difference. As a result, it was determined that PCV2 infection somewhat promoted the proliferation of lymphoid nodules around the bronchus in the lungs of pigs. In addition, it was determined that administration of PCV2b&d-ConCP vaccine or CircoFlex vaccine could somewhat promote the proliferation of lymphoid nodules. Accordingly, as the number of lymphoid nodules increases, more antigenic stimulation is received, which may induce the activation of B cells to increase the immune responses in the tissue.

The results of inflammatory cell infiltration observed around blood vessels and bronchus in the lung parenchyma were confirmed, and the results were shown in FIG. 21.

As shown in FIG. 21, the inflammatory cell infiltration was somewhat severe in the Non-challenge control group (G7) and the Challenge control group (G6), and the inflammatory cell infiltration in the Non-challenge control group (G7) was considered to be an inflammatory response caused by other pathogens naturally scattered in the farm. In the PCV2b&d-ConCP group (G4) and the CircoFlex group (G5), the inflammatory cell infiltration was relatively low, but there was no significant difference. However, as these results, it was considered that the immune response was activated by administration of the PCV2b&d-ConCP vaccine and the CircoFlex vaccine, thereby somewhat alleviating inflammation in the lungs.

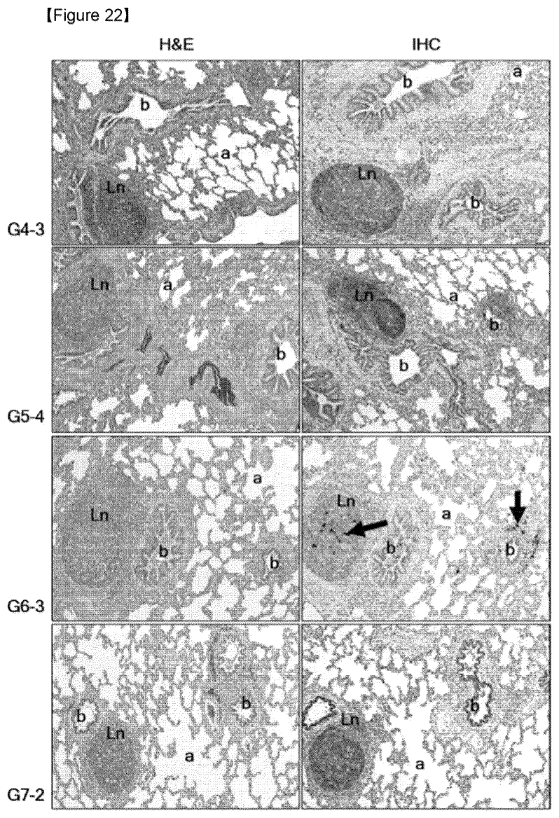

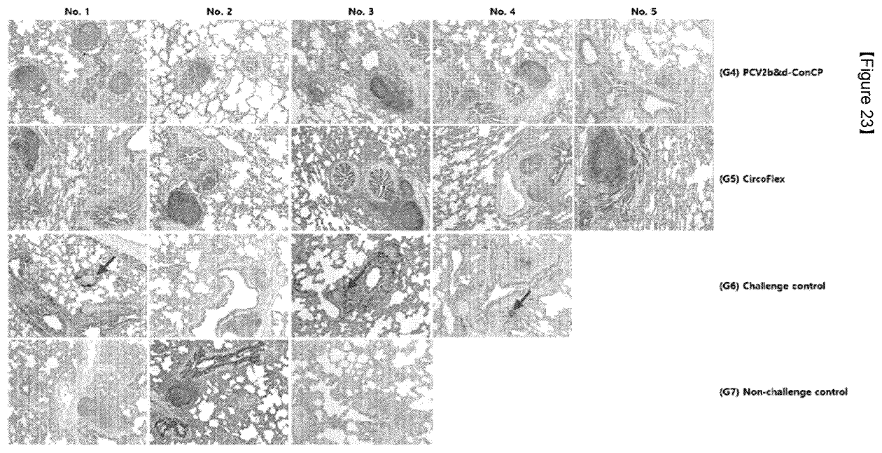

In addition, the lung tissue was immunostained for PCV2, and the results were shown in Table 21, and FIGS. 22 and 23.

TABLE 21

| | | | No | | Interstitial | Immunore |
|---|---|---|---|---|---|---|
| | | | lymphatic | Inflammatory | pneumonitis, | activity to |
| Group | ID | BALT* | nodules | cellinfiltration* | diffuse* | PCV2 |
| G4PCV2b& | 1-14-1 | 2 | 27 | 1 | 1 | — |
| d-ConCP | 1-14-2 | 2 | 26 | 1 | 1 | — |
| | 2-15-1 | 1 | 9 | 1 | 1 | — |
| | 2-15-2 | 1 | 30 | 1 | 1 | — |
| | 3-19-1 | 2 | 18 | 2 | 2 | — |
| | 3-19-2 | 1 | 12 | 1 | 1 | — |
| | 4-28-1 | 2 | 17 | 1 | 1 | — |
| | 4-28-2 | 2 | 18 | 1 | 1 | — |
| | 5-32-1 | 1 | 19 | 1 | 1 | — |
| | 5-32-2 | 1 | 29 | 1 | 1 | — |
| | MEAN | 1.5 ± | 18.5 ± | 1.1 ± 0.35 | 1.1 ± 0.32 | |
| | | 0.53 | 7.04 | | | |
| G5CircoFlex | 1-3-1 | 1 | 24 | 1 | 1 | — |
| | 1-3-2 | 1 | 16 | 1 | 1 | — |
| | 2-4-1 | 1 | 13 | 1 | 1 | — |
| | 2-4-2 | 1 | 5 | 2 | 1 | — |
| | 3-7-1 | 2 | 46 | 1 | 2 | — |
| | 3-7-2 | 2 | 41 | 2 | 2 | — |
| | 4-22-1 | 2 | 18 | 1 | 2 | — |
| | 4-22-2 | 2 | 23 | 1 | 2 | — |
| | 5-29-1 | 1 | 12 | 1 | 2 | — |
| | 5-29-2 | 2 | 28 | 1 | 2 | — |
| | MEAN | 1.5 ± | 22.6 ± | 1.2 ± 0.42 | 1.6 ± 0.52 | |
| | | 0.53 | 12.9 | | | |
| G6Challenge | 1-6-1 | 2 | 27 | 2 | 1 | 1+ |
| control | 1-6-2 | 2 | 26 | 1 | 1 | 1+ |
| | 2-8-1 | 0 | 6 | 1 | 1 | — |
| | 2-8-2 | 0 | 5 | 1 | 2 | — |
| | 3-20-1 | 0 | 4 | 2 | 2 | 3+ |
| | 3-20-2 | 1 | 12 | 2 | 3 | 3+ |
| | 4-31-1 | 2 | 9 | 3 | 3 | 3+ |
| | 4-31-2 | 2 | 8 | 3 | 4 | 2+ |
| | MEAN | 1.13 ± | 12.1 ± | 1.88 ± 0.83 | 2.13 ± 1.33 | |
| | | 0.99 | 9.22 | | | |
| G7Non- | 1-13-1 | 0 | 3 | 2 | 3 | — |
| challenge | 1-13-2 | 0 | 9 | 2 | 3 | — |
| control | 2-25-1 | 0 | 7 | 2 | 2 | — |
| | 2-25-2 | 0 | 9 | 1 | 2 | — |
| | 3-27-1 | 0 | 6 | 2 | 1 | — |
| | 3-27-2 | 0 | 5 | 2 | 1 | — |
| | MEAN | 0.0 ± | 6.5 ± | 1.83 ± 0.40 | 2.0 ± 0.89 | |
| | | 0.00 | 2.35 | | | |

As shown in Table 21, and FIGS. 22 and 23, a PCV2 positive reaction was observed only in the Challenge control group (G6), and a PCV2 positive reaction was confirmed in 3 subjects of 4 tested subjects. The PCV2 positive reaction was mainly scattered and observed in inflammatory cells (histiocytes or lymphocytes) infiltrated around the bronchus and blood vessels, in the center of lymphatic nodules, and within interstitial pneumonia. The positive reaction for PCV2 was not confirmed in the lungs of the Non-challenge control group (G7), the PCV2b&d-ConCP group (G4), and the CircoFlex group (G5).

### 3) Histological Examination of Lymph Nodes

### Changes in the Number of Secondary Lymphatic Nodules in Lymph Nodes

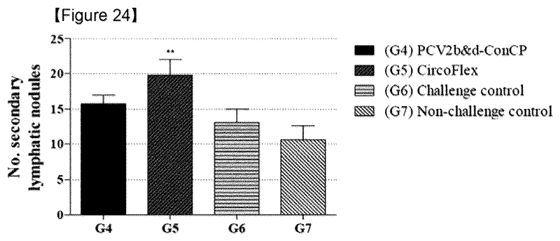

In the lymph nodes, no clear changes were observed in necrosis of lymphocytes due to PCV2 infection or the number of lymphocytes in the spread lymphatic tissue, and the number of secondary lymphatic nodules was confirmed. Cases where purulent inflammation was observed in the lymph nodes were excluded from the results. The results of comparing the number of secondary lymphatic nodules were shown in Table 22 and FIG. 24.

TABLE 22

| Group | G4 | GS | G6 | G7 |
|---|---|---|---|---|
| No. of animals (No. of | PCV2b&d-ConCP | CircoPlex | Challenge | Non-challenge |
| tissue sections examined) | | | control | control |
| | 4(8) | 4(8) | 3(6) | 3(6) |
| No. of lymphatic nodules | 15.8 ± 5.03 | 19.8 ± 8.78* | 13.0 ± 6.74 | 10.6 ± 6.65 |

Secondary lymphatic nodules were counted under ×400 magnification at 2 different areas of two different sections.
*significantly different from the uninfected control group at p < 0.05.

As shown in Table 22 and FIG. 24, it was confirmed that the number of secondary lymphatic nodules formed per unit of field of view tended to increase in the Challenge control group (G6), and to further increase even in the PCV2b&d-ConCP group (G4) and the CircoFlex group (G5). It was suggested that in the lymph nodes, since the secondary lymphatic nodules were B cell areas, an increase in secondary lymphatic nodules had activated B cells. In addition, when comparing the antibody titer test results, it was confirmed that higher antibody levels were induced in the vaccinated PCV2b&d-ConCP group (G4) and CircoFlex group (G5) than in the non-vaccinated group.

### Detection of PCV2

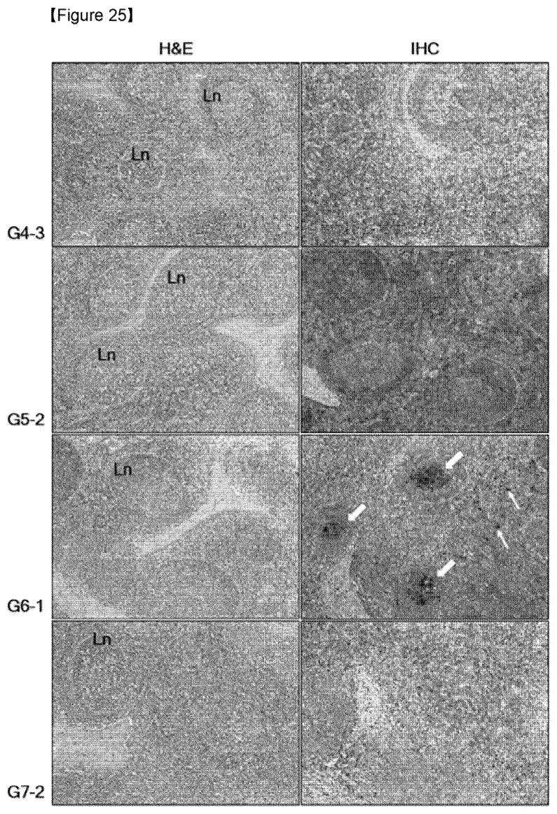

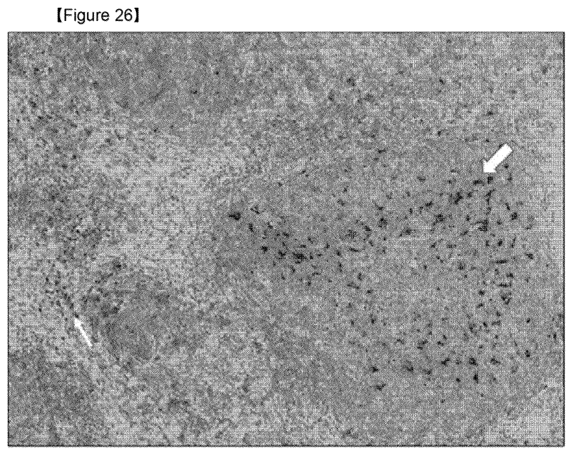

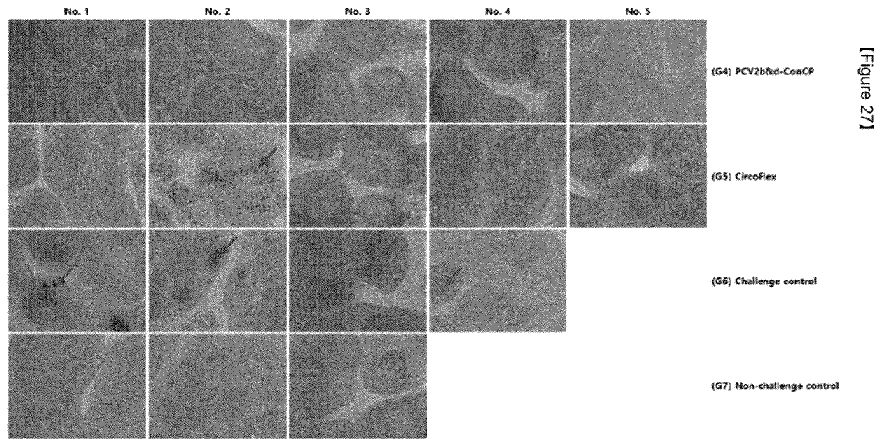

Immunohistochemical staining of PCV2 was performed in lymph nodes. The immunohistochemical staining results were shown in Table 23 and FIGS. 25 to 27.

TABLE 23

| Group | ID/Location | Lymphatic nodules | Other areas |
|---|---|---|---|
| G4PCV2b& | 1-14 | No positive cells | No positive cells |
| d-ConCP | 2-15 | No positive cells | No positive cells |
| GSCircoFlex | 3-19 | No positive cells | Some strong positive cells |
| | 4-28 | No positive cells | No positive cells |
| | 5-32 | No positive cells | No positive cells |
| | 1-3 | No positive cells | No positive cells |
| G6Challenge | 2-4 | Many strong positive cells | Quite a few strong positive cells |
| control | 3-7 | No positive cells | No positive cells |
| | 4-22 | No positive cells | No positive cells |
| G7Non- | 5-29 | No positive cells | No positive cells |
| challenge | 1-6 | Many strong positive cells | Quite a few strong positive cells |
| control | 2-8 | Many strong positive cells | Some strong positive cells |
| | 3-20 | Many strong positive cells | Quite a few strong positive cells |
| | 4-31 | Many strong positive cells | Some strong positive cells |
| | 1-13 | No positive cells | Some positive cells |
| | 2-25 | No positive cells | No positive cells |
| | 3-27 | No positive cells | Focally aggregated positive cells |

As shown in Table 23 and FIGS. 25 to 27, PCV2 antigen expression in lymph nodes was confirmed in all subjects in the Challenge control group (G6). The positive reaction for PCV2 was mainly observed strongly in the germinal center of secondary lymphatic nodules. Depending on a subject, some benign cells were observed in the spread lymphatic tissue and medullar area. However, it was determined that the local positive reactions in the spread lymphatic tissue and medullar area in some subjects were not significant because there was no significant difference from the Non-challenge control group (G7). Most subjects in the Non-challenge control group (G7), the PCV2b&d-ConCP group (G4), and the CircoFlex group (G5) did not have positive reactions in immunostaining for PCV2, but it was confirmed that the positive reaction to PCV2 occurred at the same level as the Challenge control group (G6) in one subject in the CircoFlex group (G5).

Based on these results, local PCV2 positive reactions occurred in some tissues due to the PCV2 pathogens scattered in the farm, but the results were not significant when compared to the Non-challenge control group (G7). In particular, when PCV2 positive reactions occurred in the lymph nodes of the CircoFlex group (G5) vaccinated with the commercial vaccine, it was considered that vaccination with the PCV2b&d-ConCP developed vaccine showed a more efficient defense effect against PCV2 infection.

### 2-5. Analysis of Weight Changes and Average Daily Weight Gain (ADWG) by Week of Age

### 1) Measurement of Piglet Weight by Week of Age

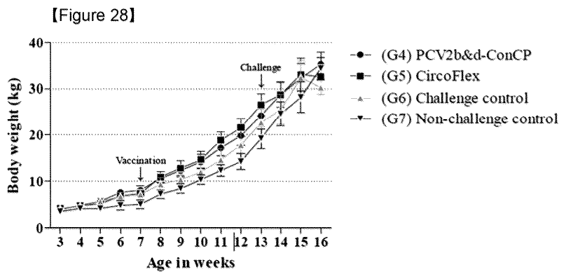

After vaccination, the average body weight of piglets by week of age was confirmed, and the results were shown in FIG. 28.

As shown in FIG. 28, 7-week-old piglets were vaccinated, and the body weights of all piglets were measured at one-week intervals during the test period. As a result of measuring the body weights of the piglets by week of age, no weight loss was shown in all piglets until the challenge (13 weeks of age), and at two weeks of the challenge (15 weeks of age), the CircoFlex group (G5) vaccinated with the commercial vaccine was maintained in the highest weight of an average of 33.0 kg. However, at 3 weeks of challenge (16 weeks of age), the Challenge control group (G6) injected with PBS had a weight loss of an average of 2.0 kg, and the CircoFlex group (G5) vaccinated with the commercial vaccine had a weight loss of an average of 0.6 kg. On the other hand, the PCV2b&d-ConCP group (G4), which showed no weight loss, gained an average of 3.0 kg. and the Non-challenge control group (G7) gained an average of 5.9 kg. At the end of the test, the average body weight was highest at 35.2 kg in the PCV2b&d-ConCP group (G4), and in the CircoFlex group (G5) at 32.44 kg, the Challenge control group (G6) at 30.25 kg, and the Non-challenge control group (G7) at 34.33 kg, differences in body weight between groups were confirmed.

### 2) Analysis of Average Daily Weight Gain (ADWG) of Piglets

The average daily weight gain of piglets after vaccination was analyzed, and the results were shown in Table 24.

TABLE 24

| | Average daily weight gain (g) | | |
|---|---|---|---|
| Groups | 0-6 DPV | 6-9 DPV | 0-9 DPV |
| PCV2b&d-ConCP | 382.4 + 42.3 | 526.7 ± 38.7 | 430.5 ± 36.3 |
| CircoFlex | 457.6 ± 41.3 | 279.0 ± 44.6 | 398.1 ± 34.4 |
| Challenge control | 368.5 ± 45.7 | 361.9 ± 82.2 | 366.3 ± 17,9 |
| Non-challenge control | 335.7 ± 26.3 | 725.4 ± 24.6 | 465.6 ± 23.1 |

As shown in Table 24, the average daily weight gain after vaccination (0)-6DPV) was 382.4 g in the PCV2b&d-ConCP group (G4), which was lower than 457.6 g in the CircoFlex group (G5). However, after the challenge (6-9DPV), the PCV2b&d-ConCP group (G4) showed a significantly higher average daily weight gain of 247.7 g than the CircoFlex group (G5) (P<0.05). Even in the average daily weight gain in the entire test period (0-9DPV), the PCV2b&d-ConCP group (G4) showed a higher level of 430.5 g than 398.1 g in the CircoFlex group (G5). Meanwhile, the Challenge control group (G6) showed the lowest average daily weight gain as 366.3 g, and the Non-challenge control group (7) showed the highest average daily weight gain as 465.6 g.

Overall, the present inventors developed PCV2b&d-ConCP, a vaccine against porcine circovirus types 2b and 2d, and effectively induced excellent IgG antibody levels and neutralizing antibody titers against both PCV2b and 2d types in pigs. This means that the PCV2b&d-ConCP vaccine of the present invention is capable of cross-protecting both PCV2b and PCV2d genotypes. In addition, even after the PCV2d challenge, significantly higher levels of IgG antibody and neutralizing antibody titers were maintained than the control group, there were shown an increase in IFN-gamma for PCV2 stimulation of PBMCs, clearance of viremia, and significantly lower levels of PCV2 DNA copy numbers in lung and lymph node tissues and nasal and rectal samples, and gross lesions of the lungs and lymph nodes were not found. In addition, there were non-detection of PCV2 antigen in histochemical staining, the highest average body weight among the test groups based on the test end date, and a significant increase in average daily weight gain, etc. As a result, this means that the PCV2b&d-ConCP vaccine may effectively prevent porcine circovirus-related diseases, and thus the PCV2b&d-ConCP vaccine of the present invention may be used in various ways in the pig farming field.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1.-11. (canceled)

12. A nucleic acid encoding a recombinant porcine circovirus type 2 (PCV2) protein represented by a nucleotide sequence represented by SEQ ID NO: 1.

13. The nucleic acid of claim 12, wherein the nucleic acid is a recombined by genes of porcine circovirus types 2b and 2d.

14. A recombinant porcine circovirus type 2 protein encoded by a nucleotide sequence represented by SEQ ID NO: 1.

15. A method for preventing, improving, or treating infectious diseases of porcine circovirus type 2, the method comprising step of administering a composition comprising a recombinant porcine circovirus type 2 protein encoded by a nucleotide sequence represented by SEQ ID NO: 1.

16. The method for preventing, improving or treating infectious diseases of porcine circovirus type 2 of claim 15, wherein the porcine circovirus type 2 is porcine circovirus type 2b or 2d.

17. The method for preventing, improving or treating infectious diseases of porcine circovirus type 2 of claim 15, wherein the infectious disease of porcine circovirus type 2 is at least one disease selected from the group consisting of porcine reproductive and respiratory syndrome (PRRS), Glasser's disease, streptococcal meningitis, salmonellosis, postweaning colibacillosis, dietetic hepatosis, and suppurative bronchopneumonia.

18. The method for preventing, improving or treating infectious diseases of porcine circovirus type 2 of claim 15, wherein the composition is administered intravenously, intramuscularly, intranasally, intra-articularly, intra-synovially, intrathecally, intrahepatically, intralesionally or intracranially.

19. The method for preventing, improving or treating infectious diseases of porcine circovirus type 2 of claim 15, wherein the composition is a vaccine composition, immunogenic composition, feed composition or feed additive composition.

20. The method for preventing, improving or treating infectious diseases of porcine circovirus type 2 of claim 15, wherein the composition is administered to a porcine.
